SEQUENCE LISTING (47 sequences)

SEQ ID NO: 1 - protein, 12 aa, Artificial Sequence
  Human outgrowth endothelial cell-binding peptides isolated from phage library
HPAIVHISPQWA

SEQ ID NO: 2 - protein, 12 aa, Artificial Sequence
  Human outgrowth endothelial cell-binding peptides isolated from phage library
QMVYGPLRSTEQ

SEQ ID NO: 3 - protein, 12 aa, Artificial Sequence
  Human outgrowth endothelial cell-binding peptides isolated from phage library
NSLTSEPLRYGG

SEQ ID NO: 4 - protein, 12 aa, Artificial Sequence
  Human outgrowth endothelial cell-binding peptides isolated from phage library
APFAHSGPLAFS

SEQ ID NO: 5 - protein, 12 aa, Artificial Sequence
  Human outgrowth endothelial cell-binding peptides isolated from phage library
TPLHPKSLMVWH

SEQ ID NO: 6 - protein, 12 aa, Artificial Sequence
  Human outgrowth endothelial cell-binding peptides isolated from phage library
SNSMHLMTMTGL

SEQ ID NO: 7 - protein, 12 aa, Artificial Sequence
  Human outgrowth endothelial cell-binding peptides isolated from phage library
SVPPRYTLTLQW

SEQ ID NO: 8 - protein, 12 aa, Artificial Sequence
  Human outgrowth endothelial cell-binding peptides isolated from phage library
TLDWTKPPLRSG

SEQ ID NO: 9 - protein, 12 aa, Artificial Sequence
  Human outgrowth endothelial cell-binding peptides isolated from phage library
ASQGYPEHRHAS

SEQ ID NO: 10 - protein, 12 aa, Artificial Sequence
  Human outgrowth endothelial cell-binding peptides isolated from phage library
HKSYLPVPSLYG

SEQ ID NO: 11 - protein, 12 aa, Artificial Sequence
  Human outgrowth endothelial cell-binding peptides isolated from phage library
QTTKLHIMDTGF

SEQ ID NO: 12 - protein, 12 aa, Artificial Sequence
  Human outgrowth endothelial cell-binding peptides isolated from phage library
ISPAPHLLTSRF

SEQ ID NO: 13 - protein, 12 aa, Artificial Sequence
  Human outgrowth endothelial cell-binding peptides isolated from phage library
HGTNQALSLLTP

SEQ ID NO: 14 - protein, 12 aa, Artificial Sequence
  Human outgrowth endothelial cell-binding peptides isolated from phage library
VLNPQTTVMPPL

SEQ ID NO: 15 - protein, 12 aa, Artificial Sequence
  Human outgrowth endothelial cell-binding peptides isolated from phage library
ATTSLTPTMANH

SEQ ID NO: 16 - protein, 12 aa, Artificial Sequence
  Human outgrowth endothelial cell-binding peptides isolated from phage library
QATGPTTPTTSG

SEQ ID NO: 17 - protein, 12 aa, Artificial Sequence
  Human outgrowth endothelial cell-binding peptides isolated from phage library
TPSLEQRTVYAK

SEQ ID NO: 18 - protein, 12 aa, Artificial Sequence
  Human outgrowth endothelial cell-binding peptides isolated from phage library
LYSASTPPDPGG

SEQ ID NO: 19 - protein, 12 aa, Artificial Sequence
  Human outgrowth endothelial cell-binding peptides isolated from phage library
FPMSSYKTYATP

SEQ ID NO: 20 - protein, 12 aa, Artificial Sequence
  Human outgrowth endothelial cell-binding peptides isolated from phage library
INTPANRNPVLG

SEQ ID NO: 21 - protein, 12 aa, Artificial Sequence
  Human outgrowth endothelial cell-binding peptides isolated from phage library
WDTNRNAASTPG

SEQ ID NO: 22 - protein, 12 aa, Artificial Sequence
  Human outgrowth endothelial cell-binding peptides isolated from phage library
SYQTLKQHLPYG

SEQ ID NO: 23 - protein, 12 aa, Artificial Sequence
  Human outgrowth endothelial cell-binding peptides isolated from phage library
HHVDSLPTLDWK

SEQ ID NO: 24 - protein, 12 aa, Artificial Sequence
  Human outgrowth endothelial cell-binding peptides isolated from phage library
KLPHQPPSAAVH

SEQ ID NO: 25 - protein, 12 aa, Artificial Sequence
  Human outgrowth endothelial cell-binding peptides isolated from phage library
SPWTSFLQWARG

SEQ ID NO: 26 - protein, 12 aa, Artificial Sequence
  Human outgrowth endothelial cell-binding peptides isolated from phage library
QFPPKLTNNSML

SEQ ID NO: 27 - protein, 12 aa, Artificial Sequence
  Human outgrowth endothelial cell-binding peptides isolated from phage library
YTDNSLGTSVGK

SEQ ID NO: 28 - protein, 12 aa, Artificial Sequence
  Human outgrowth endothelial cell-binding peptides isolated from phage library
TSLRELPAEWSR

SEQ ID NO: 29 - protein, 12 aa, Artificial Sequence
  Human outgrowth endothelial cell-binding peptides isolated from phage library
SHGKPPSRSPWT

SEQ ID NO: 30 - protein, 12 aa, Artificial Sequence
  Human outgrowth endothelial cell-binding peptides isolated from phage library
YNLGQLEAQITS

SEQ ID NO: 31 - protein, 12 aa, Artificial Sequence
  Human outgrowth endothelial cell-binding peptides isolated from phage library
ITLSATKGAAPS

SEQ ID NO: 32 - protein, 12 aa, Artificial Sequence
  Human outgrowth endothelial cell-binding peptides isolated from phage library
SPPPSNAGSHHV

SEQ ID NO: 33 - protein, 12 aa, Artificial Sequence
  Human outgrowth endothelial cell-binding peptides isolated from phage library
THPPNPSVSIGG

SEQ ID NO: 34 - protein, 12 aa, Artificial Sequence
  Human outgrowth endothelial cell-binding peptides isolated from phage library
MPTSSTAPPPLI

SEQ ID NO: 35 - protein, 12 aa, Artificial Sequence
  Human outgrowth endothelial cell-binding peptides isolated from phage library
ANYFSSPIKHAT

SEQ ID NO: 36 - protein, 12 aa, Artificial Sequence
  Human outgrowth endothelial cell-binding peptides isolated from phage library
HPPHNMHLPAFS

SEQ ID NO: 37 - protein, 12 aa, Artificial Sequence
  Human outgrowth endothelial cell-binding peptides isolated from phage library
MPTLTRAPHTAC

SEQ ID NO: 38 - protein, 12 aa, Artificial Sequence
  Human outgrowth endothelial cell-binding peptides isolated from phage library
LPRKTPDYLQTR

SEQ ID NO: 39 - protein, 12 aa, Artificial Sequence
  Consensus sequence for Human outgrowth endothelial cell-binding peptides isolated from phage library
SPTPSXPPSAGG

SEQ ID NO: 40 - protein, 1569 aa, Homo sapiens
MVQRWLLLSCCGALLSAGLANTSYTSPGLQRLKDSPQTAPDKGQCSTWGAGHFSTFDHHVYDFSGTCNYIFAATCKDAFPSFSVQLRRGPDGSISRIIVELGASVVTVSEAIISVKDIGVISLPYTSNGLQITPFGQSVRLVAKQLELELEVVWGPDSHLMVLVERKYMGQMCGLCGNFDGKVTNEFVSEEGKFLEPHKFAALQKLDDPGEICTFQDIPSTHVRQAQHARGCTQLLTLVAPECSVSKEPFVLSCQADVAAAPQPGPQNSSYATLSEYSRQCSMVGQPVALRSPGLCSVGQCPANQVYQECGSACVKTCSNSEHSCSSSCTFGCFCPEGTDLNDLSNNHTCVPVTQCPCVLHGAMYAPGEVTIAACQTCRCTLGRWVCTERPCPGHCSLEGGSFVTTFDARPYRFHGTCTYILLQSPQLPEDGALMAVYDKSGVSHSETSLVAVVYLSRQDKIVISQDEVVTNNGEAKWLPYKTRNITVFRQTSTHLQMATSFGLELVVQLRPIFQAYVTVGPQFRGQTRGLCGNFNGDTTDDFTTSMGIAEGTASLFVDSWRAGNCPDALERETDPCSMSQLNKVCAETHCSMLLRTGTVFERCHATVNPAPIYKRCMYQACNYEETFPHICAALGDYVHACSLRGVLLWGWRSSVDNCTIPCTGNTTFSYNSQACERTCLSLSDRATECHHSAVPVDGCNCPDGTYLNQKGECVRKAQCPCILEGYKFILAEQSTVINGITCHCINGRLSCPQRLQMFLASCQAPKTFKSCSQSSENKFGAACAPTCQMLATGVACVPTKCEPGCVCAEGLYENAYGQCVPPEECPCEFSGVSYPGGAELHTDCRTCSCSRGRWACQQGTHCPSTCTLYGEGHVITFDGQRFVFDGNCEYILATDVCGVNYSQPTFKILTENVICGNSGVTCSRAIKIFLGGLSVVLADRNYTVTGEEPHVQLGVTPGALSLVVDISIPGRYNLTLIWNRHMTILIRIARASQDPLCGLCGNFNGNMKDDFETRSRYVASSELELVNSWKESPLCGDVSFVTDPCSLNAFRRSWAERKCSVINSQTFATCHSKVYHLPYYEACVRDACGCDSGGDCECLCDAVAAYAQACLDKGVCVDWRTPAFCPIYCGFYNTHTQDGHGEYQYTQEANCTWHYQPCLCPSQPQSVPGSNIEGCYNCSQDEYFDHEEGVCVPCMPPTTPQPPTTPQLPTTGSRPTQVWPMTGTSTTIGLLSSTGPSPSSNHTPASPTQTPLLPATLTSSKPTASSGEPPRPTTAVTPQATSGLPPTATLRSTATKPTVTQATTRATASTASPATTSTAQSTTRTTMTLPTPATSGTSPTLPKSTNQELPGTTATQTTGPRPTPASTTGPTTPQPGQPTRPTATETTQTRTTTEYTTPQTPHTTHSPPTAGSPVPSTGPVTATSFHATTTYPTPSHPETTLPTHVPPFSTSLVTPSTHTVITPTHAQMASSASNHSAPTGTIPPPTTLKATGSTHTAPPITPTTSGTSQAHSSFSTNKTPTSLHSHTSSTHHPEVTPTSTTSITPNPTSTRTRTPMAHTNSATSSRPP

SEQ ID NO: 41 - protein, 217 aa, Homo sapiens
MAKAYPQYYRKPSVVKRMPAMLTGLCQGCGTRQVVFTSDPHKSYLPVQFQSPDKAETQRGDPSVISVNGTDFTFRSAGVLLLVVDPCSVPFRLTEKTVFPLADVSRIEEYLKTGIPPRSIVLLSTRGEIKQLNISHLLVPLGLAKPAHLYDKGSTIFLGFSGNFKPSWTKLFTSPAGQGLGVLEQFIPLQLDEYGCPRATTVRRRDLELLKQASKAH

SEQ ID NO: 42 - protein, 389 aa, Homo sapiens [sequence omitted]

SEQ ID NO: 43 - protein, 346 aa, Mus muscularis
MVGGGGKRRTAGAGPQCEKTVEVKKSKFSEADVSSDLRKEVENLYKLSLPEDFYHFWKFCEELDPEKPADALATSLGLRLVGPYDILAGKHKMKKKPTGLNCNLHWRFYYDPPEFQTIIIGDNKTQYHMGYFRDSPDELPVYVGTNEAKKNCIIIQNGDNVFAAIKLFLMKKLKEVTDRKKISILKNIDEKLTEAARKLGYSLEQRTVKMRQRDKKVVTKTFHGAGLVVPVDKNDVGYRELPETDADLKRICKAVVDAASDEERLKAFAPIQEMMTFVQFANDECDYGMGLELGMDLFCYGSHYFHKVAGQLLPLAYNLLKRDLFAKIIEDHLASRSEENIDQLAG

SEQ ID NO: 44 - protein, 199 aa, Homo sapiens
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 45 - protein, 21531 aa, Homo sapiens
MLKPSGLPGSSSPTRSLMTGSRSTKATPEMDSGLTGATLSPKTSTGAIVVTEHTLPFTSPDKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTDTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWASVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPSTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGTSPAGTSPGSPEVSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLKDPEYAGHKLGIWDDFIPKFGKAAHMRELPLLSPPQDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVTWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPETVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAMANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSVAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHVILTTDEDTTAIEAMHPSTSTAATNVETTCSGHGSQSSVLTDSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSFGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSPLTPGQVKTTEVLDASAEPETSSPPSLSSTSVEILATSEVTTDTEKIHPFPNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLATSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLTPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSALPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISSEVITRLSTSPIKAESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILATTEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLATSKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMSSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRTHIPDSDQSTMSPDIITEVITRLSSSSMMSESTQMTITTQKSSPGATAQSTLTLATTTAPLARTHSTVPPRFLHSEMTTLMSRSPENPSWKSSPFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSTPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASRQMASTILTEMMITTPYVFPDVPETTSSLATSLGAETSTALPRTTPSVLNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPKTTPKFSHSKSDTTLPVAITSPGPEASSAVSTTTISPDMSDLVTSLVPSSGTDTSTTFPTLSETPYEPETTVTWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTGTSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSAMAVDAICTHRPDPEDLGLDRERLYWELSNLTNGIQELGPYTLDRNSLYVNGFTHRSSMPTTSTPGTSTVDVGTSGTPSSSPSPTAAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTTSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGLDREQLYWELSQLTNGIKELGPYTLDRNSLYVNGFTHWIPVPTSSTPGTSTVDLGSGTPSSLPSPTAAGPLLVPFTLNFTITNLQYEEDMHHPGSRKFNTTERVLQGLLGPMFKNTSVGLLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMRHPGSRKFNTTERVLQGLLKPLFKSTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTAGPLLVPFTLNFTITNLQYEEDMHRPGSRKFNTTERVLQGLLSPIFKNSSVGPLYSGCRLTSLRPEKDGAATGMDAVCLYHPNPKRPGLDREQLYWELSQLTHNITELGPYSLDRDSLYVNGFTHQNSVPTTSTPGTSTVYWATTGTPSSFPGHTEPGPLLIPFTFNFTITNLHYEENMQHPGSRKFNTTERVLQGLLKPLFKNTSVGPLYSGCRLTSLRPEKDGAATGMDAVCLYHPNPKRPGLDREQLYWELSQLTHNITELGPYSLDRDSLYVNGFTHQNSVPTTSTPGTSTVYWATTGTPSSFPGHTEPGPLLIPFTFNFTITNLHYEENMQHPGSRKFNTTERVLQGLLKPLFKNTSVGPLYSGCRLTLLRPEKHEAATGVDTICTHRVDPIGPGLDRERLYWELSQLTNSITELGPYTLDRDSLYVNGFNPRSSVPTTSTPGTSTVHLATSGTPSSLPGHTAPVPLLIPFTLNFTITNLHYEENMQHPGSRKFNTTERVLQGLLKPLFKNTSVGPLYSGCRLTLLRPEKHEAATGVDTICTHRVDPIGPGLELYWELSLTIELGPYTLDRSLYVNGFTHSPTTSTPGTSTVGTSGTPSSPTSAGPLLVPFTLNFTITNLQYEEDMHHPGSRKFNTTERVLQGLLGPMFKNTSVGLLYSGCRLTLLRPEKNGAATGMDAICSHRLDPKSPGLDREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTAGPLLVPFTLNFTITNLQYEEDMHRPGSRKFNATERVLQGLLSPIFKNSSVGPLYSGCRLTSLRPEKDGAATGMDAVCLYHPNPKRPGLDREQLYWELSQLTHNITELGPYSLDRDSLYVNGFTHQSSMTTTRTPDTSTMHLATSRTPASLSGPTTASPLLVLFTINCTITNLQYEEDMRRTGSRKFNTMESVLQGLLKPLFKNTSVGPLYSGCRLTLLRPKKDGAATGVDAICTHRLDPKSPGLNREQLYWELSKLTNDIEELGPYTLDRNSLYVNGFTHQSSVSTTSTPGTSTVDLRTSGTPSSLSSPTIMPLLPFTNTITNLMPGSRKFNTTERVLQGLLRPLFKNTSVSSLYSGCRLTLLRPEKDGAATRVDAACTYRPDPKSPGLDREQLYWELSQLTHSITELGPYTLDRVSLYVNGFNPRSSVPTTSTPGTSTVHLATSGTPSSLPGHTPLLPFTNTITNLMPGSRKFNTTERVLQGLLKPLFRNSSLEYLYSGCRLASLRPEKDSSAMAVDAICTHRPDPEDLGLDRERLYWELSNLTNGIQELGPYTLDRNSLYVNGFTHRSSGLTTSTPWTSTVDLGTSGTPSPVPSPTTAGPLLVPFTLNFTITNLQYEEDMHRPGSRRFNTTERVLQGLLTPLFKNTSVGPLYSGCRLTLLRPEKQEAATGVDTICTHRVDPIGPGLDRERLYWELSQLTNSITELGPYTLDRDSLYVNGFNPWSSVPTTSTPGTSTVHLATSGTPSSLPGHTAPVPLLIPFTLNFTITDLHYEENMQHPGSRKFNTTERVLQGLLKPLFKSTSVGPLYSGCRLTLLRPEKHGAATGVDAICTLRLDPTGPGLDRERLYWELSQLTNSVTELGPYTLDRDSLYVNGFTHRSSVPTTSIPGTSAVHLETSGTPASLPGHTAPGPLLVPFTLNFTITNLQYEEDMRHPGSRKFSTTERVLQGLLKPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTTLRPKKDGAATKVDAICTYRPDPKSPGLDREQLYWELSQLTHSITELGPYTQDRDSLYVNGFTHRSSVPTTSIPGTSAVHLETSGTPASLPGHTAPGPLLVPFTLNFTITNLQYEEDMRHPGSRKFNTTERVLQGLLKPLFKSTSVGPLYSGCRLTLLRPEKRGAATGVDTICTHRLDPLNPGLDREQLYWELSKLTRGIIELGPYLLDRGSLYVNGFTHRTSVPTTSTPGTSTVDLGTSGTPFSLPSPAPLLPFTNTITNLMPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTPLLPFTNTITNLMPGSRKFNTTEVLQGLLPFKNSVGLYSGCRLTLREKGAATGDAICHPKPGLELYWELSLTIELGPYTLDRSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTSLRSEKDGAATGVDAICTHRVDPKSPGVDREQLYWELSQLTNGIKELGPYTLDRNSLYVNGFTHQTSAPNTSTPGTSTVGTSGTPSSPTSAGPLLVPFTLNFTITNLQYEEDMHHPGSRKFNTTERVLQGLLGPMFKNTSVGLLYSGCRLTLLRPEKNGATTGMDAICTHRLDPKSPGLELYWELSLTIELGPYTLDRSLYVNGFTHSPTTSTPGTSTVGTSGTPSSPTPLLPFTNTITNLMPGSRKFNTTERVLQGLLKPLFRNSSLEYLYSGCRLASLRPEKDSSAMAVDAICTHRPDPEDLGLDRERLYWELSNLTNGIQELGPYTLDRNSLYVNGFTHRSSMPTTSTPGTSTVDVGTSGTPSSSPSPTTAGPLLIPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTTSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGLELYWELSLTIELGPYTLDRSLYVNGFTHSPTTSTPGTSTVGTSGTPSSPTPLLPFTNTITNLMPGSRKFNTTERVLQGLLRPVFKNTSVGPLYSGCRLTLLRPKKDGAATKVDAICTYRPDPKSPGLDREQLYWELSQLTHSITELGPYTQDRDSLYVNGFTHRSSVPTTSIPGTSAVHLETTGTPSSFPGHTEPGPLLIPFTFNFTITNLRYEENMQHPGSRKFNTTERVLQGLLTPLFKNTSVGPLYSGCRLTLLRPEKQEAATGVDTICTHRVDPIGPGLDRERLYWELSQLTNSITELGPYTLDRDSLYVDGFNPWSSVPTTSTPGTSTVHLATSGTPSPLPGHTAPVPLLIPFTLNFTITDLHYEENMQHPGSRKFNTTERVLQGLLKPLFKSTSVGPLYSGCRLTLLRPEKHGAATGVDAICTLRLDPTGPGLDRERLYWELSQLTNSITELGPYTLDRDSLYVNGFNPWSSVPTTSTPGTSTVHLATSGTPSSLPGHTTAGPLLVPFTLNFTITNLKYEEDMHCPGSRKFNTTERVLQSLHGPMFKNTSVGPLYSGCRLTLLRSEKDGAATGVDAICTHRLDPKSPGLELYWELSLTIELGPYTLDRSLYVNGFTHSPTTSTPGTSTVGTSGTPSSPTPLLPFTNTITNLMPGSRKFNTTEVLQGLLPFKNSVGLYSGCRLTLREKGAATGDAICHPKPGLELYWELSLTNSITELGPYTLDRDSLYVNGFTHRSSMPTTSIPGTSAVHLETSGTPASLPGHTAPGPLLVPFTLNFTITNLQYEEDMRHPGSRKFNTTERVLQGLLKPLFKSTSVGPLYSGCRLTLLRPEKRGAATGVDTICTHRLDPLNPGLELYWELSLTIELGPYTLDRSLYVNGFTHSPTTSTPGTSTVGTSGTPSSPTPLLPFTNTITNLMPGSRKFNTTEVLQGLLPFKNSVGLYSGCRLTLREKGAATGDAICHPKPGLELYWELSLTIELGPYTLDRSLYVNGFHPRSSVPTTSTPGTSTVHLATSGTPSSLPGHTAPVPLLIPFTLNFTITNLHYEENMQHPGSRKFNTTERVLQGLLGPMFKNTSVGLLYSGCRLTLLRPEKNGAATGMDAICSHRLDPKSPGLELYWELSLTIELGPYTLDRSLYVNGFTHSPTTSTPGTSTVGTSGTPSSPTPLLPFTNTITNLMPGSRKFNTTEVLQGLLPFKNSVGLYSGCRLTLREKGAATGDAICHPKPGLELYWELSLTIELGPYTLDRSLYVNGFTHQNSVPTTSTPGTSTVYWATTGTPSSFPGHTEPGPLLIPFTFNFTITNLHYEENMQHPGSRKFNTTERVLQGLLTPLFKNTSVGPLYSGCRLTLLRPEKQEAATGVDTICTHRVDPIGPGLELYWELSLTIELGPYTLDRSLYVNGFTHSPTTSTPGTSTVGTSGTPSSPTPLLPFTNTITNLMPGSRKFNTTEVLQGLLPFKNSVGLYSGCRLTLREKGAATGDAICHPKPGLELYWELSLTIELGPYTLDRSLYVNGFTHRSSVPTTSSPGTSTVHLATSGTPSSLPGHTAPVPLLIPFTLNFTITNLHYEENMQHPGSRKFNTTERVLQGLLKPLFKSTSVGPLYSGCRLTLLRPEKHGAATGVDAICTLRLDPTGPGLELYWELSLTIELGPYTLDRSLYVNGFTHSPTTSTPGTSTVGTSGTPSSPTPLLPFTNTITNLMPGSRKFNTTEVLQGLLPFKNSVGLYSGCRLTLREKGAATGDAICHPKPGLELYWELSLTIELGPYTLDRSLYVNGFTHRTSVPTTSTPGTSTVHLATSGTPSSLPGHTAPVPLLIPFTLNFTITNLQYEEDMHRPGSRKFNTTERVLQGLLSPIFKNSSVGPLYSGCRLTSLRPEKDGAATGMDAVCLYHPNPKRPGLDREQLYCELSQLTHNITELGPYSLDRDSLYVNGFTHQNSVPTTSTPGTSTVYWATTGTPSSFPGHTPLLPFTNTITNLMPGSRKFNTTEVLQGLLPFKNSVGLYSGCRLTLREKGAATGDAICHPKPGLELYWELSLTIELGPYTLDRSLYVNGFTHWSSGLTTSTPWTSTVDLGTSGTPSPVPSPTTAGPLLVPFTLNFTITNLQYEEDMHRPGSRKFNATERVLQGLLSPIFKNTSVGPLYSGCRLTLLRPEKQEAATGVDTICTHRVDPIGPGLELYWELSLTIELGPYTLDRSLYVNGFTHSPTTSTPGTSTVGTSGTPSSPTPLLPFTNTITNLMPGSRKFNTTEVLQGLLPFKNSVGLYSGCRLTLREKGAATGDAICHPKPGLELYWELSLTIELGPYTLDRSLYVNGFTHRSFGLTTSTPWTSTVDLGTSGTPSPVPSPTTAGPLLVPFTLNFTITNLQYEEDMHRPGSRKFNTTERVLQGLLTPLFRNTSVSSLYSGCRLTLLRPEKDGAATRVDAVCTHRPDPKSPGLELYWELSLTIELGPYTLDRSLYVNGFTHSPTTSTPGTSTVGTSGTPSSPTPLLPFTNTITNLMPGSRKFNTTEVLQGLLPFKNSVGLYSGCRLTLREKGAATGDAICHPKPGLELYWELSLTIELGPYTLDRSLYVNGFTHWIPVPTSSTPGTSTVDLGSGTPSSLPSPTTAGPLLVPFTLNFTITNLQYGEDMGHPGSRKFNTTERVLQGLLGPIFKNTSVGPLYSGCRLTSLRSEKDGAATGVDAICIHHLDPKSPGLELYWELSLTIELGPYTLDRSLYVNGFTHSPTTSTPGTSTVGTSGTPSSPTPLLPFTNTITNLMPGSRKFNTTEVLQGLLPFKNSVGLYSGCRLTLREKGAATGDAICHPKPGLELYWELSLTIELGPYTLDRSLYVNGFTHQTFAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATRVDAVCTHRPDPKSPGLELYWELSLTIELGPYTLDRSLYVNGFTHSPTTSTPGTSTVGTSGTPSSPTAPVPLLIPFTLNFTITNLHYEENMQHPGSRKFNTTERVLQGLLKPLFKSTSVGPLYSGCRLTLLRPEKHGAATGVDAICTLRLDPTGPGLDRERLYWELSQLTNSVTELGPYTLDRDSLYVNGFTQRSSVPTTSIPGTSAVHLETSGTPASLPGHTAPGPLLVPFTLNFTITNLQYEVDMRHPGSRKFNTTERVLQGLLKPLFKSTSVGPLYSGCRLTLLRPEKRGAATGVDTICTHRLDPLNPGLDREQLYWELSKLTRGIIELGPYLLDRGSLYVNGFTHRNFVPITSTPGTSTVHLGTSETPSSLPRPIVPGPLLVPFTLNFTITNLQYEEAMRHPGSRKFNTTERVLQGLLRPLFKNTSIGPLYSSCRLTLLRPEKDKAATRVDAICTHHPDPQSPGLNREQLYWELSQLTHGITELGPYTLDRDSLYVDGFTHWSPIPTTSTPGTSIVNLGTSGIPPSLPETTPLLPFTNTITNLMPGSRKFNTTERVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMHRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGHYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGSRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMKHLLSPLFQRSSLGARYTGCRVIALRSVKNGAETRVDLLCTYLQPLSGPGLPIKQVFHELSQQTHGITRLGPYSLDKDSLYLNGYNEPGLDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGLITCLICGVLVTTRRRKKEGEYNVQQQCPGYYQSHLDLEDLQ

SEQ ID NO: 46 - protein, 2337 aa, Homo sapiens
MVRFGDELGGRYGGPGGGERARGGGAGGAGGPGPGGLQPGQRVLYKQSIAQRARTMALYNPIPVKQNCFTVNRSLFVFSEDNVVRKYAKRITEWPPFEYMILATIIANCIVLALEQHLPDGDKTPMSERLDDTEPYFIGIFCFEAGIKIIALGFVFHKGSYLRNGWNVMDFVVVLTGILATAGTDFDLRTLRAVRVLRPLKLVSGIPSLQVVLKSIMKAMVPLLQIGLLLFFAILMFAIIGLEFYMGKFHKACFPNSTDAEPVGDFPCGKEAPARLCEGDTECREYWPGPNFGITNFDNILFAILTVFQCITMEGWTDILYNTNDAAGNTWNWLYFIPLIIIGSFFMLNLVLGVLSGEFAKERERVENRRAFLKLRRQQQIERELNGYLEWIFKAEEVMLAEEDRNAEEKSPLDVLKRAATKKSRNDLIHAEEGEDRFADLCAVGSPFARASLKSGKTESSSYFRRKEKMFRFFIRRMVKAQSFYWVVLCVVALNTLCVAMVHYNQPRRLTTTLYFAEFVFLGLFLTEMSLKMYGLGPRSYFRSSFNCFDFGVIVGSVFEVVWAAIKPGSSFGISVLRALRLLRIFKVTKYWSSLRNLVVSLLNSMKSIISLLFLLFLFIVVFALLGMQLFGGQFNFQDETPTTNFDTFPAAILTVFQILTGEDWNAVMYHGIESQGGVSKGMFSSFYFIVLTLFGNYTLLNVFLAIAVDNLANAQELTKDEEEMEEAANQKLALQKAKEVAEVSPMSAANISIAARQQNSAKARSVWEQRASQLRLQNLRASCEALYSEMDPEERLRFATTRHLRPDMKTHLDRPLVVELGRDGARGPVGGKARPEAAEAPEGVDPPRRHHRHRDKDKTPAAGDQDRAEAPKAESGEPGAREERPRPHRSHSKEAAGPPEARSERGRGPGPEGGRRHHRRGSPEEAAEREPRRHRAHRHQDPSKECAGAKGERRARHRGGPRAGPREAESGEEPARRHRARHKAQPAHEAVEKETTEKEATEKEAEIVEADKEKELRNHQPREPHCDLETSGTVTVGPMHTLPSTCLQKVEEQPEDADNQRNVTRMGSQPPDPNTIVHIPVMLTGPLGEATVVPSGNVDLESQAEGKKEVEADDVMRSGPRPIVPYSSMFCLSPTNLLRRFCHYIVTMRYFEVVILVVIALSSIALAAEDPVRTDSPRNNALKYLDYIFTGVFTFEMVIKMIDLGLLLHPGAYFRDLWNILDFIVVSGALVAFAFSGSKGKDINTIKSLRVLRVLRPLKTIKRLPKLKAVFDCVVNSLKNVLNILIVYMLFMFIFAVIAVQLFKGKFFYCTDESKELERDCRGQYLDYEKEEVEAQPRQWKKYDFHYDNVLWALLTLFTVSTGEGWPMVLKHSVDATYEEQGPSPGYRMELSIFYVVYFVVFPFFFVNIFVALIIITFQEQGDKVMSECSLEKNERACIDFAISAKPLTRYMPQNRQSFQYKTWTFVVSPPFEYFIMAMIALNTVVLMMKFYDAPYEYELMLKCLNIVFTSMFSMECVLKIIAFGVLNYFRDAWNVFDFVTVLGSITDILVTEIANNFINLSFLRLFRAARLIKLLRQGYTIRILLWTFVQSFKALPYVCLLIAMLFFIYAIIGMQVFGNIALDDDTSINRHNNFRTFLQALMLLFRSATGEAWHEIMLSCLSNQACDEQANATECGSDFAYFYFVSFIFLCSFLMLNLFVAVIMDNFEYLTRDSSILGPHHLDEFIRVWAEYDPAACGRISYNDMFEMLKHMSPPLGLGKKCPARVAYKRLVRMNMPISNEDMTVHFTSTLMALIRTALEIKLAPAGTKQHQCDAELRKEISVVWANLPQKTLDLLVPPHKPDEMTVGKVYAALMIFDFYKQNKTTRDQMQQAPGGLSQMGPVSLFHPLKATLEQTQPAVLRGARVFLRQKSSTSLSNGGAIQNQESGIKESVSWGTQRTQDAPHEARPPLERGHSTEIPVGRSGALAVDVQMQSITRRGPDGEPQPGLESQGRAASMPRLAAETQPVTDASPMKRSISTLAQRPRGTHLCSTTPDRPPPSQASSHHHHHRCHRRRDRKQRSLEKGPSLSADMDGAPSSAVGPGLPPGEGPTGCRRERERRQERGRSQERRQPSSSSSEKQRFYSCDRFGGREPPKPKPSLSSHPTSPTAGQEPGPHPQGSGSVNGSPLLSTSGASTPGRGGRRQLPQTPLTPRPSITYKTANSSPIHFAGAQTSLPAFSPGRLSRGLSEHNALLQRDPLSQPLAPGSRIGSDPYLGQRLDSEASVHALPEDTLTFEEAVATNSGRSSRTSYVSSLTSQSHPLRRVPNGYHCTLGLSSGGRARHSYHHPDQDHWC

SEQ ID NO: 47 - protein, 4 aa, Artificial Sequence
  linker sequence
GGGS

(19) United States
(12) Patent Application Publication
(10) Pub. No.: US 2010/0119476 A1
(43) Pub. Date: May 13, 2010

(54) SELECTION OF PEPTIDES SPECIFIC FOR HUMAN BLOOD OUTGROWTH ENDOTHELIAL CELLS

(75) Inventors: Cam Patterson, Chapel Hill, NC (US); Egede Aitsebaomo, Durham, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: University of North Carolina at Chapel hill, Chapel Hill, NC (US)

(21) Appl. No.: 12/612,289

(22) Filed: Nov. 4, 2009

Related U.S. Application Data
(63) Continuation-in-part of application No. 12/530,137.
(60) Provisional application No. 60/892,987, filed on Mar. 5, 2007.

(51) Int. Cl. A61K 38/20; A61P 9/00; A61P 9/10

(52) U.S. Cl. 424/85.2

(57) ABSTRACT

Provided herein are compositions and methods for binding outgrowth endothelial cells (OEC). The compositions consist of peptide ligands capable of binding OEC with high affinity and specificity. The compositions of the invention include peptides set forth in SEQ ID NO:1-38 and variants and derivatives thereof. Compositions also include the nucleotide sequences encoding the peptides of the invention. The compositions find use in methods for the isolation of OEC and for the recruitment and retention of OEC to sites of therapeutic interest. Methods for the identification and isolation of other peptides capable of binding OEC are also provided.

### CROSS-REFERENCE TO RELATED APPLICATIONS

This is a continuation-in-part of co-pending U.S. patent application Ser. No. 12/530,137, mailed Sep. 4, 2009, which is a U.S. National Stage Entry of PCT/US2008/055874, filed Mar. 5, 2008, which claims the benefit of U.S. Provisional Application No. 60/892,987, filed Mar. 5, 2007, each of which is hereby incorporated in its entirety by reference herein.

### FEDERALLY SPONSORED RESEARCH OR DEVELOPMENT

This invention was made with United States Government support under Grant Nos. R01 HL61656, K08 HL085293, and R01 HL065619 awarded by the National Institutes of Health. The United States Government has certain rights in the invention.

### REFERENCE TO SEQUENCE LISTING SUBMITTED ELECTRONICALLY

The official copy of the sequence listing is submitted electronically via EFS-Web as an ASCII formatted sequence listing with a file named “380112_SequenceListing.txt”, created on Oct. 22, 2009, and having a size of 223 kilobytes and is filed concurrently with the specification. The sequence listing contained in this ASCII formatted document is part of the specification and is herein incorporated by reference in its entirety.

### FIELD OF THE INVENTION

The present invention is directed to compositions and methods for treating vascular disease and ischemia by enhancing the retention of outgrowth endothelial cells at the site of vascular injury.

### BACKGROUND OF THE INVENTION

Cardiovascular disease and its associated maladies, dysfunctions and complications are a principal cause of disability and the chief cause of death in the United States. One factor contributing to cardiovascular disease is atherosclerosis. Atherosclerosis has been generally recognized as the leading health care problem both with respect to mortality and health care costs.

Atherosclerosis is a disease characterized by the deposition of fatty substances, primarily cholesterol, and subsequent fibrosis in the inner layer of an artery, resulting in plaque deposition on the inner surface of the arterial wall and degeneration. If allowed to progress, atherosclerosis can cause narrowing and obstruction of the lumen of the artery resulting in diminished or occluded blood flow. This can lead to ischemia or infarction of the predominantly affected organ or anatomical region, such as the brain, heart, intestine, or extremities.

Angiogenesis is the process of new blood vessel development from preexisting vasculature. Angiogenesis is a normal process in growth and development, as well as in wound healing. It can occur during coronary artery disease, peripheral artery disease and stroke when there is insufficient blood supply and oxygen to the tissues. Vasculogenesis is the process of blood vessel formation from endothelial progenitor cells (EPC) that differentiate in situ.

Until recently, vasculogenesis was thought to be limited to embryologic development. However, the discovery of circulating endothelial progenitor cells has provided evidence that postnatal vasculogenesis also occurs in adults. Progenitor cell-based regenerative strategies offer new perspectives in cell therapies and tissue engineering for achieving an effective revascularization of ischemic or injured tissues. Cultures from peripheral blood contain cells termed early-EPC that share some endothelial but also monocytic characteristics and exhibit a restricted capacity of expansion. Another cell population isolated from peripheral blood cultures is called late-EPC or blood outgrowth endothelial cells (BOEC) that have a cobblestone morphology and have high proliferative capacity.

### SUMMARY OF THE INVENTION

Compositions and methods for binding outgrowth endothelial cells (OEC) are provided. The compositions comprise peptide ligands capable of binding OEC with high affinity and specificity. The compositions of the invention include peptides set forth in Table 1 and variants and derivatives thereof. Compositions also include the nucleotide sequences encoding the peptides of the invention. The nucleotide sequences can be used in DNA constructs or expression cassettes for transformation and expression in mammals.

The compositions find use in methods for the isolation of OEC and for the recruitment and retention of OEC to sites of therapeutic interest. Methods for use of the compositions in cell therapies including angiogenesis, blood vessel repair, ischemic tissue repair, and therapeutic revascularization are provided. The compositions can be used in combination with biomedical devices. Methods for the identification and isolation of other peptides capable of binding OEC are also provided.

### BRIEF DESCRIPTION OF THE DRAWINGS

FIG. 1A provides a schematic representation of the biopanning procedure for selection of peptide ligands that bind specifically to OEC.

FIG. 1B shows a concentration dependence of four selected phage clones (SEQ ID NO:7, white bar; SEQ ID NO:17, dark gray bar; SEQ ID NO:32, light gray bar; SEQ ID NO:37, gray striped bar), binding to human blood OEC (HBOEC); Homogeneous phage at the indicated concentrations were biopanned on HBOEC for 1 hour at 4° C. Values showing the mean±standard deviation from triplicate determinations.

FIG. 2 exhibits binding of ligands containing TP(S/T/G) motif (from left to right, SEQ ID NO:17, 19, 21, 13, 15, and 16). 1.10¹¹ of homogeneous phage were biopanned on HBOEC for 1 hour at 4° C. All determinations were performed at least three times and the data are shown as mean±standard deviation.

FIG. 3 displays cell specificity of ligands selected by biopanning on HBOEC (SEQ ID NO:7, white bar; SEQ ID NO:17, dark gray bar; SEQ ID NO:32, light gray bar; SEQ ID NO:37, gray striped bar). Specificity determinations used inputs of 1.10¹¹ phage and incubations of 1 hour at 4° C. Values showing the mean±standard deviation of triplicate determinations.

FIG. 4 demonstrates the functional characterization on a peptide level. HBOEC were incubated with the phage display-identified peptides in a dose-dependent manner and proliferation was monitored over time. A. TPS-peptide. B. SYQ-peptide. C. SWD-peptide.

FIG. 5 represents the quantification of the tube formation ability of the HBOEC-synthetic peptide complexes as a function of peptide concentration. HBOEC were incubated with the phage display-identified peptides in a dose-dependent manner and tube formation was monitored after 6 hours.

FIG. 6 represents the quantification of the migration ability of the HBOEC-synthetic peptide complexes as a function of peptide concentration. HBOEC were incubated with the phage display-identified peptides in a dose-dependent manner and migration in a Boyden chamber assay was monitored after 12 hours.

FIG. 7 represents the quantification of response to VEGF. HBOEC were incubated with the phage display-identified peptides in a dose-dependent manner and their response to VEGF was monitored after 96 hours. A. TPS-peptide. B. SYQ-peptide. C. SWD-peptide.

FIG. 8 represents the functional characterization of peptides immobilized to a biomaterial surface and the design of peptide modified bioactive materials. A. Hexylmethacrylate (HMA), methylmethacrylate (MMA), and methacrylic acid (MAA) were used to synthesize methacrylic terpolymers via free radical polymerization reaction. B. Peptide sequences were attached using chain transfer chemistry with terminal cysteine residue serving as a chain transfer agent. C. Bar graph quantifying cell attachment of HBOEC on peptide modified HMA:MMA:MAA terpolymer films. RGE-modified terpolymer surfaces were used as a negative control; Fibronectin (FN) coated wells were used as a positive control. SYQ and TPS denote HBOEC specific peptides identified by phage display selection. The cell-adhesive RGD peptide was included in the study as well. The peptide density in all materials is similar, around 2 μmol peptide per gram terpolymer as determined by amino acid analysis. D. Bar graph quantifying cell attachment of HUVEC on peptide modified HMA:MMA:MAA terpolymer films.

FIG. 9 is a schematic of the EPC capture technology for spontaneous endogenous endothelialization. The bioengineered prosthetic surface is a biocompatible terpolymer matrix with covalently-coupled, high-affinity peptide ligands that bind EPC from the circulation.

FIG. 10 is a schematic of the vascular construct for EPC capture in a porcine carotid artery model. A. Peptide-modified methacrylic terpolymer in the form of a rectangular sheet measuring 6.0 mm×6.0 mm×0.5 mm is suspended in the axis of a hollow cylindrical hub constructed of woven polyester, commercially-available vascular graft (length=2.5 cm, diameter=1 cm). This combination is held in mid-axial position by smooth polyethylene sutures anchoring the prosthesis and extending radially to the suspended biomaterial. B. Cross-sectional view.

FIG. 11 is a schematic of the vascular surgery for EPC capture in a porcine carotid artery model. A. Longitudinal arteriotomy incision of the external carotid artery. B. The implant is inserted into the lumen of the external carotid artery distal to the arteriotomy incision. The prosthesis is fixed to the arterial wall with 6-0 polyethylene sutures in a simple continuous pattern, anchoring the edge of the cylindrical hub around the circumference of the arterial wall. C. The longitudinal arteriotomy incision is sutured with 6-0 polypropylene sutures in a continuous pattern.

FIG. 12. Human blood outgrowth endothelial cells express IL-11Rα and rhIL-11 treatment stimulates HBOEC to effect signaling through STAT-3. A) HBOEC were stained with anti human IL-11 receptor Ab-Alexa488 and analyzed by flow cytometry. HBOEC brightly stained with α IL-11 receptor compared to mouse IgG isotype antibody control. B) HBOEC were treated with IL-11 (25 ng/ml) of rhIL-11 for 1, 2.5, 5, 10, 20, 30, 60 and 120 min. Cell lysates were analyzed by western blotting with anti-phospho STAT-3 (Tyr705) Ab and anti-STAT (79D7) Ab. Recombinant human IL-11 induced STAT-3 phosphorylation in HBOEC at the indicated time.

FIG. 13. Recombinant human interleukin-11 acts as chemoattractant to HBOECs and induces sprouting of HBOC spheroids. A) Effect of rhIL-11 on HBOEC migration in Boyden chamber assay. HBOEC were suspended in serum starved medium and plated in the upper chamber. Media, rhIL-11 or VEGF solutions at the indicated concentrations were added in the lower chamber. After 4 h 30 minutes of incubation, cells that had migrated to the lower surface were stained and counted. Each bar in the graph represents the mean of three independent experiments. Unpaired t-test for rhIL-11 vs PBS treated mice, two-tailed ***=P<0.0001. B) Images of collagen embedded HBOEC spheroids shows sprouting after 24 hrs of media, rhIL-11 or VEGF treatment. C) Recombinant human IL-11 induces 11-fold increase in the cumulative sprout length, compared to media treated control. D) Treatment with rhIL-11 results in 8-folds increase in the number of sprouts per spheroid. Each bar in the graph represents the mean of three independent experiments. Unpaired t-test for rhIL-11 vs PBS treated mice, two-tailed ***=P<0.0001.

FIG. 14. Recombinant human IL-11 treatment leads to in vivo mobilization of CD34+/VEGFR2+ mononuclear cells. A SV129 mice were implanted with osmotic pumps loaded with either PBS or rhIL-11 for 3 days. Mononuclear cells from the mouse blood were analyzed by flow cytometry and profiled according to their forward and side scatter. B) PBS treated mice showed fewer CD34+/VEGFR2+ mononuclear cells in the R2 quadrant compared with rhIL-11 treated mice (C) under the same experimental condition. D) CD34+/VEGFR2+ mononuclear cells mobilization peaked on day 3 after rhIL-11 treatment and treated mice show a 20-fold higher number of CD34+/VEGFR2+ mononuclear cells in the peripheral blood compared with PBS control mice on day 3. Each bar is mean 35 SEM of 6 mice. Unpaired t-test for rhIL-11 vs PBS treated mice, two-tailed *=P<0.05.

FIG. 15. Recombinant human IL-11 treated mice show increased collateral vessel blood flow recovery and increased perfusion after femoral artery ligation. Sv129 mice were pre-treated with rhIL-11 or PBS for 3 days prior to femoral artery ligation—thus day 0 on graph is equivalent to day 3 after inception of rhIL-infusion. A) Recombinant human IL-11 treated mice showed blood flow recovery and plantar vessel perfusion compared to PBS control mice. B) Graph showing ratio of perfusion rate in ligated/non-ligated hindlimb. Recombinant human IL-11 treated mice show significantly increased perfusion rate from day 4 to day 8 after femoral artery ligation. n=9 per data point. C) Mice treated with rhIL-11 have more adductor perfusion that was significant 4 days after femoral artery ligation. Increased adductor perfusion did not significant on day 6 and 8. Region marked with in white is used for calculating total perfusion rate in adductor muscle. Color scale show relation between color and units of perfusion rate.

FIG. 16. Blood flow recovery in rhIL-11 treated mice is linked to hindlimb functional recovery. After femoral artery ligation, animals were individually inspected for foot appearance score [index of ischemia: 0, normal; 1-5, cyanosis or loss of nail(s), where the score is dependent on the number of nails affected; 6-10, partial or complete atrophy of digit(s), where the score reflects number of digits affected; 11, partial atrophy of forefoot]. Hindlimb use scores (index of muscle function) will be obtained: 0, normal; 1, no toe flexion; 2, no plantar flexion; 3, dragging foot. Recombinant human IL-11 treated mice have better functional recovery of both hindlimb use A, and hindlimb appearance B when compared to PBS control mice.

FIG. 17. Immunohistochemical analysis showing histologic evidence of adductor vessel remodeling in rhIL-11 treated mice. A) Cyano-Masson-Elastin staining of anterior and posterior gracilis muscle. B) Graph showing that rhIL-11 treated mice showed 3-fold increase in luminal diameter of adductor collateral vessel compared to PBS control. N=9. Recombinant human IL-11 treated mice have more α-smooth muscle than PBS control (C) and (D).

FIG. 18. Recombinant human IL-11 treatment leads to homing of CD34+/VEGFR2+ mononuclear cells toward collateral vessels. Graph showing increased peripheral monocytes (A) and platelet (B) in rhIL-11 treated mice. Histologic analysis of gracilis muscle stained with a CD11b Ab-FITC (C) and CD34+/VEGFR2+ mononuclear cells (D) at 20× magnification showing homing of macrophages and CD34+/VEGFR2+ mononuclear cells toward adductor collateral vessel undergoing remodeling.

### DETAILED DESCRIPTION OF THE INVENTION

### Overview

The present invention provides novel peptides for use in therapeutic methods employing cell therapy to treat vascular diseases, including atherosclerosis and heart disease. The invention is further directed to a method for inducing angiogenesis or neovascularization in a mammal by administering to the mammal an effective amount of the peptides of the invention in combination with OECs. The compositions may further employ a population of endothelial precursor cells, cardiac microvascular endothelial cells (CMECs), young bone marrow cells, stem cells, embryonic stem cell lines or hematopoietic stem cells to treat vascular disease or ischemia.

The invention is based on studies demonstrating the effectiveness of outgrowth endothelial cells (OEC) for treating vascular disease and ischemia by promoting neovascularization and re-endothelialization. Neovascularization refers to the development of new blood vessels from endothelial precursor cells by any means, such as by vasculogenesis, angiogenesis, or the formation of new blood vessels from endothelial precursor cells that link to existing blood vessels. Angiogenesis is the process by which new blood vessels grow from the endothelium of existing blood vessels in a developed animal. Angiogenesis is essential for wound healing and for reproduction. Re-endothelialization refers to the homing of circulating endothelial precursor cells to sites of intimal injury such as occurs in atherosclerotic plaques.

Endothelial precursor cells such as OECs circulate in the blood and selectively migrate, or “home,” to sites of active angiogenesis (see U.S. Pat. No. 5,980,887, Isner et al., the contents of which are incorporated herein by reference in their entirety). OECs (also referred to as are circulating bone marrow-derived endothelial cells and late EPCs) are closer to mature endothelial cells in phenotype but show surprising proliferative, migrating, and tube-forming capabilities. OECs exhibit the typical “cobblestone” morphology of endothelial cells. These cells incorporate acetylated low-density lipoprotein (LDL) and are uniformly positive for vWF, P1H12, thrombomodulin, flk-1, VE-cadherin, PECAM-1, CD34, CD36, and integrin αᵥ. They are uniformly negative for monocyte marker CD14.

Such endothelial precursor cells are capable of homing to sites of cardiac angiogenic induction and homing to sites of intimal injury to facilitate re-endothelialization. These cells can restore and stimulate cardiac angiogenesis in an aging host, for example, by healing injured vascular tissues, reducing the size of atherosclerotic lesions, stimulating angiogenesis, generating cardiac myocytes and promoting formation of new blood vessels and new endothelial tissues.

The present invention provides compositions and methods for identifying, purifying, and characterizing OECs, as well as improving the therapeutic efficacy of OECs in treating vascular disorders and injury.

### Compositions

Peptides capable of specific binding to outgrowth endothelial cells (OEC) with high affinity are provided. The peptides of the invention comprise those set forth in SEQ ID NO:1-38 and 40-46, as well as variants and derivatives thereof. Some of the peptides are characterized by the presence of consensus motifs. These consensus motifs are underlined in some of the peptides listed in Table 1.

All of the peptides set forth in Table 1 contain 12 amino acids. However, it is recognized that the peptides may contain fewer than 12 amino acids or more than 12 amino acids. The peptides of the invention comprise at least 6, at least 7, at least 8, at least 9 at least 10, at least 11, at least 12, up to at least about 40 amino acids. That is, the peptides may comprise at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least about 40 amino acids, at least about 50, at least about 60, at least about 70, at least about 80, at least about 100, at least about 150, at least about 200, at least about 250, at least about 300, or up to the full length of the protein from which the 12-amino acid peptide listed in Table 1 was identified.

As indicated above, the peptides may contain at least one consensus motif. The motifs include PLR, PPR, TP, TPT, TPS, TPG, PPS, and MPT.

The term “peptide” broadly refers to an amino acid chain that includes naturally occurring amino acids, synthetic amino acids, genetically encoded amino acids, non-genetically encoded amino acids, and combinations thereof. Peptides can include both L-form and D-form amino acids.

Representative non-genetically encoded amino acids include but are not limited to 2-aminoadipic acid; 3-aminoadipic acid; β-aminopropionic acid; 2-aminobutyric acid; 4-aminobutyric acid (piperidinic acid); 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

Representative derivatized amino acids include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-im-benzylhistidine.

Biologically active variants of the peptides of the invention are also encompassed by the present invention. Such variants should retain binding activity to OEC, particularly the ability to specifically bind OEC. Binding activity can be measured by methods in the art. For example, see the experimental section of the present application. Preferably, the variant has at least the same activity as the native molecule. The activity can also be associated with the affinity and/or specificity of OEC binding, or can be associated with particular downstream in vivo activities such as improved perfusion, decreased neointimal formation, decreased thromboses, and greater capillary density when administered to a subject as described elsewhere herein.

Suitable biologically active variants can be fragments and derivatives. By “fragment” is intended a peptide consisting of only a part of the intact peptide sequence and structure, and can be a C-terminal deletion or N-terminal deletion of amino acids or deletions at both the C- and N-terminal ends. By “derivatives” is intended any suitable modification of a binding peptide or peptide fragment encompassing any change in amino acid residues, so long as the binding activity is retained.

Peptide variants will generally have at least 50%, at least 60%, at least 70%, preferably at least 80%, more preferably about 90% to 95% or more, about 96%, about 97%, and most preferably about 98%, about 99% or more amino acid sequence identity to the amino acid sequence of the reference peptide molecule. A variant may differ by as few as 3, 2, or even 1 amino acid residue. Methods for determining identity between sequences are well known in the art. See, for example, the ALIGN program (Dayhoff (1978) in Atlas of Protein Sequence and Structure 5:Suppl. 3 (National Biomedical Research Foundation, Washington, D.C.) and programs in the Wisconsin Sequence Analysis Package, Version 8 (available from Genetics Computer Group, Madison, Wis.), for example, the GAP program. For purposes of optimal alignment of the two sequences, the contiguous segment of the amino acid sequence of the variant may have additional amino acid residues or deleted amino acid residues with respect to the amino acid sequence of the reference molecule. The contiguous segment used for comparison to the reference amino acid sequence will comprise at least twelve (12), at least about 13, at least about 14, about 15, about 16, about 17, about 18, about 19, about 20, about 25, about 30, at least about 35 or more amino acids. Corrections for increased sequence identity associated with inclusion of gaps in the variant's amino acid sequence can be made by assigning gap penalties. Methods of sequence alignment are well known in the art. However, when calculating the percent identity of a sequence compared to an amino acid sequence consisting of any one of SEQ ID NO:1-38 or 40-46, the percent identity is calculated across the entirety of any one of SEQ ID NO:1-38 or 40-46, and gaps are typically not allowed.

When considering percentage of amino acid sequence identity, some amino acid residue positions may differ as a result of conservative amino acid substitutions, which do not affect properties of protein function. In these instances, percent sequence identity may be adjusted upwards to account for the similarity in conservatively substituted amino acids. Such adjustments are well known in the art. See, for example, Meyers and Miller (1988) Computer Applic. Biol. Sci. 4:11-17.

For example, preferably, conservative amino acid substitutions may be made. A “nonessential” amino acid residue is a residue that can be altered without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). See, for example, Sambrook J., and Russell, D. W. (2001) Molecular Cloning: A Laboratory Manual. (Cold Spring Harbor Laboratory Press, Cold Spring Harbor, N.Y.) and Innis, et al. (1990) PCR Protocols: A Guide to Methods and Applications (Academic Press, NY).

The peptides of the invention can be subject to various changes, substitutions, insertions, and deletions where such changes provide for certain advantages in its use. Thus, the term “peptide” encompasses any of a variety of forms of peptide derivatives including, for example, amides, conjugates with proteins, cyclone peptides, polymerized peptides, conservatively substituted variants, analogs, fragments, chemically modified peptides, and peptide mimetics. Any peptide that has desired binding characteristics can be used in the practice of the present invention.

By “binds specifically” or “specific binding” is intended that the peptides bind to OEC but do not bind to other cell types. In some embodiments, a peptide that binds specifically to OEC binds at least 10%, 20%, 30%, 40%, 50%, 60%, 70%, 80%, 90%, 100%, 200%, 300%, 400%, 500%, or a higher percentage more than the peptide binds to an appropriate control such as, for example, a different cell type.

One aspect of the invention pertains to isolated nucleic acid molecules comprising nucleotide sequences encoding binding peptides or biologically active portions thereof. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

Nucleic acid molecules that are fragments of these binding peptide encoding nucleotide sequences are also encompassed by the present invention. By “fragment” is intended a portion of the nucleotide sequence encoding a binding peptide. Nucleic acid molecules that are fragments of a binding peptide nucleotide sequence comprise at least about 15, 20, 50, 75, 100 contiguous nucleotides. By “contiguous” nucleotides is intended nucleotide residues that are immediately adjacent to one another.

The skilled artisan will further appreciate that changes can be introduced by mutation into the nucleotide sequences of the invention thereby leading to changes in the amino acid sequence of the encoded binding peptides, without altering the binding specificity or affinity of the peptides. Thus, variant isolated nucleic acid molecules can be created by introducing one or more nucleotide substitutions, additions, or deletions into the corresponding nucleotide sequence disclosed herein, such that one or more amino acid substitutions, additions or deletions are introduced into the encoded peptide. Mutations can be introduced by standard techniques, such as site-directed mutagenesis and PCR-mediated mutagenesis. Such variant nucleotide sequences are also encompassed by the present invention.

### Uses

The peptides of the invention find use in methods for the isolation, recruitment, and retention of OEC. Thus, the peptides of the invention can be used to sequester OEC cells at therapeutic sites of interest and in cell-based therapeutic revascularization approaches to ischemic disease and endothelial injury. “Therapeutic sites of interest” include areas where angiogenesis is desired, areas of ischemic injury, areas of organ transplantation, areas of vascular injury, and the like. Thus, strategies can enhance the number of endothelial cells in the vessel wall following injury and limit complications such as thrombosis, vasospasm, and neointimal formation, through reconstitution of a luminal barrier and cellular secretion of paracrine factors.

The peptides of the invention can be introduced at a therapeutic site of interest. Any method for introducing the peptides at the site may be employed. In the same manner, a therapeutic site of interest can be seeded with at least one peptide of the invention to aid in the selection and retention of OEC at the site. By “seeding” or “seeded” is intended any means for introducing the peptides at the site. Such methods include injection, infusion, and the like. It is recognized that the peptides may be introduced at the site to capture and retain endogenous OEC at the site of interest. Alternatively, peptides with OEC bound may be introduced at the therapeutic site. In the same manner, the peptides may be delivered by gene delivery techniques. That is, the peptides may be expressed at a site of interest by vectors designed to express the peptides in a mammal.

### Vascular Diseases

The vascular diseases treated by the present invention are vascular diseases of mammals. The word mammal means any mammal. Some examples of mammals include, for example, pet animals, such as dogs and cats; farm animals, such as pigs, cattle, sheep, and goats; laboratory animals, such as mice and rats; primates, such as monkeys, apes, and chimpanzees; and humans. In some embodiments, humans are preferably treated by the methods of the invention.

According to the invention, endothelial cells within normal vascular tissues change as they grow older, exhibit reduced angiogenesis, reduced capacity for re-endothelization and lose their ability to communicate with other cells by secreting signaling agents. These changes can lead to a diminished capacity for blood vessel formation, a reduction in blood flow to the associated organ or system, and an inability to recover from injuries or diseases that adversely affect blood vessels.

Accordingly, the invention relates to methods for treating endothelial dysfunction, or a vascular condition, or a circulatory condition, such as a condition associated with loss, injury or disruption of the vasculature within an anatomical site or system. The term “vascular condition” or “vascular disease” refers to a state of vascular tissue where blood flow is, or can become, impaired.

Many pathological conditions can lead to vascular diseases that are associated with alterations in the normal vascular condition of the affected tissues and/or systems. Examples of vascular conditions or vascular diseases to which the methods of the invention apply are those in which the vasculature of the affected tissue or system is senescent or otherwise altered in some way such that blood flow to the tissue or system is reduced or in danger of being reduced. Examples of vascular conditions that can be treated with the compositions and methods of the invention include atherosclerosis, preeclampsia, peripheral vascular disease, erectile dysfunction, cancers, renal failure, heart disease, and stroke. Vascular, circulatory or hypoxic conditions to which the methods of the invention apply also include those associated with, but not limited to, maternal hypoxia (e.g., placental hypoxia, preeclampsia), abnormal pregnancy, peripheral vascular disease (e.g., arteriosclerosis), transplant accelerated arteriosclerosis, deep vein thrombosis, erectile dysfunction, cancers, renal failure, stroke, heart disease, sleep apnea, hypoxia during sleep, female sexual dysfunction, fetal hypoxia, smoking, anemia, hypovolemia, vascular or circulatory conditions which increase risk of metastasis or tumor progression, hemorrhage, hypertension, diabetes, vasculopathologies, surgery (e.g., per-surgical hypoxia, post-operative hypoxia), Raynaud's disease, endothelial dysfunction, regional perfusion deficits (e.g., limb, gut, renal ischemia), myocardial infarction, stroke, thrombosis, frost bite, decubitus ulcers, asphyxiation, poisoning (e.g., carbon monoxide, heavy metal), altitude sickness, pulmonary hypertension, sudden infant death syndrome (SIDS), asthma, chronic obstructive pulmonary disease (COPD), congenital circulatory abnormalities (e.g., Tetralogy of Fallot) and Erythroblastosis (blue baby syndrome). In particular embodiments, the invention is a method of treating loss of circulation or endothelial dysfunction in an individual.

Thus, the invention is directed to compositions useful in a method of treating diseases such as stroke, atherosclerosis, acute coronary syndromes including unstable angina, thrombosis and myocardial infarction, plaque rupture, both primary and secondary (in-stent) restenosis in coronary or peripheral arteries, transplantation-induced sclerosis, peripheral limb disease, intermittent claudication and diabetic complications (including ischemic heart disease, peripheral artery disease, congestive heart failure, retinopathy, neuropathy and nephropathy), or thrombosis.

In some embodiments, the vascular condition or vascular disease arises from damaged myocardium. As used herein “damaged myocardium” refers to myocardial cells that have been exposed to ischemic conditions. These ischemic conditions may be caused by a myocardial infarction, or other cardiovascular disease. The lack of oxygen causes death of the cells in the surrounding area, leaving an infarct that can eventually scar.

Preferably, damaged myocardium is treated with the methods and compositions of the invention before damage occurs (e.g. when damage is suspected of occurring) or as quickly as possible after damage occurs. Hence, the methods and compositions of the invention are advantageously employed on aged heart tissues that are in danger of ischemia, heart attack or loss of blood flow. The methods and compositions of the invention are also advantageously employed on recently damaged myocardium and on not so recently damaged myocardium.

As used herein “recently damaged myocardium” refers to myocardium that has been damaged within one week of treatment being started. In a preferred embodiment, treatment with the compositions of the invention is initiated within three days of myocardial damage. In a further preferred embodiment, treatment is initiated within 12 hours of myocardial damage.

In one embodiment, the present invention may be used to enhance blood vessel formation in ischemic tissue, i.e., a tissue having a deficiency in blood as the result of an ischemic disease. Such tissues can include, for example, muscle, brain, kidney and lung. lschemic diseases include, for example, cerebrovascular ischemia, renal ischemia, pulmonary ischemia, limb ischemia, ischemic cardiomyopathy and myocardial ischemia.

The methods of the present invention may also be used to treat blood vessel injuries that result in denuding of the endothelial lining of the vessel wall. For example, primary angioplasty is becoming widely used for the treatment of acute myocardial infarction. In addition, endovascular stents are becoming widely used as an adjunct to balloon angioplasty. Stents are useful for rescuing a sub-optimal primary result as well as for diminishing restenosis. To date, however, the liability of the endovascular prosthesis has been its susceptibility to thrombotic occlusion in approximately 3% of patients with arteries 3.3 mm or larger. If patients undergo stent deployment in arteries smaller than this size, the incidence of sub-acute thrombosis is even higher. Sub-acute thrombosis is currently prevented only by the aggressive use of anticoagulation. The combination of vascular intervention and intense anticoagulation creates significant risks with regard to peripheral vascular trauma at the time of the stent/angioplasty procedure. Acceleration of re-endothelialization by administration of stents, implants, or biomedical devices coated with a peptide capable of attracting OECs to a patient undergoing, or subsequent to, angioplasty and/or stent deployment can stabilize an unstable plaque and prevent re-occlusion.

### Pharmaceutical Compositions

The invention encompasses pharmaceutical compositions comprising the peptides of the invention. It is recognized that the pharmaceutical composition may contain a plurality of a single binding peptide or mixtures of peptides. Likewise when the peptides are used to coat implants, a single peptide may be used or a combination of peptides may be used. Pharmaceutical compositions formulated with a mixture of at least one binding peptide can be made by methods known in the art. See Remington's Pharmaceutical Sciences (18^{th} ed.; Mack Pub. Co.: Eaton, Pa., 1990), herein incorporated by reference. The pharmaceutical composition is administered to supply a desired therapeutic dose to promote a desired therapeutic response of the peptide to the therapeutic area. By “desired therapeutic response” is intended an improvement in the condition or in the symptoms associated with the condition, and the promotion of angiogenesis.

The compositions of this invention will be formulated in a unit dosage such as a solution, suspension or emulsion, in association with a pharmaceutically acceptable carrier. Such carriers are inherently nontoxic and nontherapeutic. Examples of such carriers are saline, Ringer's solution, dextrose solution, and Hanks' solution. Nonaqueous carriers such as fixed oils and ethyl oleate may also be used. The vehicle may contain minor amounts of additives such as substances that enhance chemical stability, including buffers and preservatives.

Suitable methods of delivery of the pharmaceutical composition include, but are not limited to, gel formulations, viscous solutions, sustained-release formulations, implant delivery systems, such as pumps, and the like. Such delivery systems allow for the controlled and concentrated delivery of the peptide(s) to a therapeutic site. The exact formulation employed will depend on the type of application that is desired.

A pharmaceutically effective amount of a pharmaceutical composition of the invention is administered to a subject. By “pharmaceutically effective amount” is intended an amount that is useful in the treatment of a disease or condition, where treatment can be for a therapeutic purpose as noted herein above. In this manner, a pharmaceutically effective amount of the composition will administer a therapeutically effective dose or amount of the binding peptide to the subject in need of treatment. By “therapeutically effective dose or amount” or “effective amount” is intended an amount of the binding peptide that, when administered brings about a positive therapeutic response with respect to angiogenesis, blood vessel repair, ischemic tissue repair, and therapeutic revascularization. In some embodiments of the invention, the therapeutically effective dose is in the range from about 0.1 μg/kg to about 100 mg/kg body weight, about 0.001 mg/kg to about 50 mg/kg, about 0.01 mg/kg to about 30 mg/kg, about 0.1 mg/kg to about 25 mg/kg, about 1 mg/kg to about 20 mg/kg, about 3 mg/kg to about 15 mg/kg, about 5 mg/kg to about 12 mg/kg, about 7 mg/kg to about 10 mg/kg or any range of value therein. It is recognized that the method of treatment may comprise a single administration of a therapeutically effective dose or multiple administrations of a therapeutically effective dose.

It is understood that the effective amount may vary depending on the nature of the effect desired, frequency of treatment, any concurrent treatment, the health, weight of the recipient, and the like. See, e.g., Berkow et al., eds., Merck Manual, 16th edition, Merck and Co., Rahway, N.J. (1992); Goodman et al., eds., Goodman and Gilman's The Pharmacological Basis of Therapeutics, 8th edition, Pergamon Press, Inc., Elmsford, N.Y. (1990); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston (1985), Katzung, Basic and Clinical Phamacology, Appleton and Lange, Norwalk, Conn. (1992), which references and references cited therein, are entirely incorporated herein by reference.

The pharmaceutical composition may be contained in a pharmaceutically-acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. The use of such media and agents for delivering cells is well known in the art. Except insofar as any conventional media or agent is incompatible with the cells or polypeptides provided herein, use thereof in the compositions is contemplated. Supplementary active compounds can also be incorporated into the compositions.

A pharmaceutical composition of the invention is formulated to be compatible with its intended route of administration. Examples of routes of administration include intravenous, intraarterial, intracoronary, parenteral, subcutaneous, subdermal, subcutaneous, intraperitoneal, intraventricular infusion, infusion catheter, balloon catheter, bolus injection, direct application to tissue surfaces during surgery, or other convenient routes. Solutions or suspensions used for such administration can include other components such as sterile diluents like water for dilution, saline solutions, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The composition can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHORE EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions to accompany the cellular suspensions can be prepared by incorporating an active compound (e.g., a PDGF B polypeptide or PDGF AB protein) in the required amount in an appropriate solvent with a selected combination of ingredients, followed by filter sterilization. Generally, dispersions are prepared by incorporating an active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

It is especially advantageous to formulate the cells and/or compositions in dosage unit form for ease of administration and uniformity of dosage. Dosage unit form as used herein refers to physically discrete units suited as unitary dosages for the subject to be treated. Each unit can then contain a predetermined quantity of the peptides and/or cells and other components calculated to produce the desired therapeutic effect in association with the required pharmaceutical carrier.

The pharmaceutical compositions may be co-administered with other agents known to mobilize hematopoietic precursors, with agents known to promote the differentiation of embryonic endothelial cell precursors, or with agents believed to induce angiogenesis, for example, 3-hydroxy-3-methylglutaryl coenzyme A reductase inhibitors (statins), endothelial growth factor, vascular endothelial growth factor (VEGF), fibroblast growth factor (FGF), granulocyte-colony stimulating factor (G-CSF), granulocyte-macrophage-colony stimulating factor (GM-CSF), stem cell factor (SCF), interleukin-3 (Tong et al., Exptl. Hematol. 22:1016-1024, 1994; de Revel et al., Blood 83:3795-3799, 1994; Schots et al., Bone Marrow Transplantation 17:509-515, 1996), and angiopoietin (Takehara et al., Cell 49:415-422, 1987; Suri et al., Cell 87:1171-1180, 1996).

### Implants, Stents and Biomedical Devices

It is recognized that the peptides can be administered to therapeutic sites alone or alternatively may be attached to an acceptable implant, stent, or other biomedical device. In this manner, the implant may be coated with peptides. In some embodiments, the peptides will be attached to the implants. Likewise, when the peptides are administered directly and when they are used as coatings for implants, OEC may be attached to the peptides.

The term “implant” generally refers to a structure that is introduced into a human or animal body to restore a function of a damaged tissue or to provide a new function. An implant device can be created using any biocompatible material. Representative implants include but are not limited to: vascular prostheses, artificial heart valves, hip endoprostheses, artificial joints, jaw or facial implants, tendon and ligament replacements, skin replacements, bone replacements, bone graft devices, stents, shunts, nerve growth guides, intraocular lenses, and the like. Implants further comprise scaffolds that merely hold the peptides and/or bound OEC at therapeutic sites of interest. In general, tissue scaffolds are small, porous, implants made of specially designed biomaterials that support a therapeutic site and assist the body in growing new, functional tissue. If the scaffold is degradable, when the damaged or lost tissue has been successfully replaced by new tissue, the scaffold will have completely resorbed.

An “implantable” device is the device, which is adapted for permanent or temporary insertion into or application against a tissue of a mammal such as, for example, a human. Examples of implantable devices or components include, but are not limited to, an artificial heart, cardiac pacer leads, automatic implantable cardiodefibrilator leads, a prosthetic heart valve, a cardiopulmonary bypass membrane, a ventricular assist device, an annuloplasty ring, a dermal graft, a vascular graft, a vascular, cardiovascular, or structural stent, a catheter, a guide wire, a vascular or cardiovascular shunt, a dura mater graft, a cartilage graft, a cartilage implant, a pericardium graft, a ligament prosthesis, a tendon prosthesis, a urinary bladder prosthesis, a pledget, a suture, a permanently in-dwelling percutaneous device, an artificial joint, an artificial limb, a bionic construct (i.e. one of the aforementioned devices or components comprising a microprocessor or other electronic component), and a surgical patch.

Implants are made of a variety of materials that are known in the art and include but are not limited to: a polymer or a mixture of polymers including, for example, biodegradable plastics, polylactic acid, polyglycolic acid, polylactic acid-polyglycolic acid copolymers, polyanhidrides, polyorthoesters, polystyrene, polycarbonate, nylon, PVC, collagen (including, for example, processed collagen such as cross-linked collagen), glycosaminoglycans, hyaluronic acid, alginate, silk, fibrin, cellulose, and rubber; plastics such as polyethylene (including, for example, high-density polyethylene (HDPE)), PEEK (polyetheretherketone), and polytetrafluoroethylene; metals such as titanium, titanium alloy, stainless steel, and cobalt chromium alloy; metal oxides; non-metal oxides; silicone; bioactive glass; ceramic material such as, for example, aluminum oxide, zirconium oxide, and calcium phosphate; other suitable materials such as demineralized bone matrix; and combinations thereof. The term “polymer” as used herein refers to any of numerous natural and synthetic compounds of usually high molecular weight consisting of up to millions of repeated linked units, each a relatively simple molecule.

Synthetic grafts useful in the present invention may be composed of any material suitable for this purpose. To be suitable, a graft must be suturable to the host vessel, durable, and impervious to blood loss at implantation. Typically, synthetic grafts are pretreated prior to implantation, e.g., preclotted with autologous blood, or are coated with partially hydrolyzed proteins during manufacture. Preferred materials for the vascular grafts used in accord with the subject methods include polyethylene terephthalate and polytetrafluoroethylene (PTFE). In one embodiment, the synthetic vascular graft is composed of polyethylene terephthalate, which may be knit or woven. It is within the contemplation of this invention that these or other synthetic substances can be chemically modified to enhance their susceptibility to colonization by circulating endothelial precursor cells.

Thus, the present invention provides methods for preparing an implant to be surgically placed into a patient wherein the device is coated with at least one binding peptide. Methods for attaching peptides to implants are generally known in the art, i.e., by the use of bovine serum albumin, by the use of acrylic acid coupling, bromoalkylation, etc. The peptides may be applied by dipping, spraying, or brushing a solution containing the peptide onto the implant. See, e.g., Harris et al. (2004) Biomaterials 25: 4135-4148 and U.S. patent application Ser. No. 10/644,703, filed Aug. 19, 2003 and published on May 6, 2004 with Publication No. 20040087505.

In one embodiment of the invention, the peptide mediates OEC cell attachment to the surface of an implant. By enhancing OEC adhesion, the peptides of the invention can accelerate healing, accelerate angiogenesis and improve the function of the implanted device. Implants can be coated with the peptides of the invention before implantation. Likewise in some embodiments, the implants will be coated with peptides bound to OECs for implantation. This method is referred to herein as “seeding” the OECs on the implantation device.

There are multiple techniques known in the art for the seeding of selected cells to an implantation device (see, for example, U.S. Pat. Nos. 5,674,722; 5,785,965; and 5,766,584). Typically, the implantation device is incubated in vitro, optionally with rotation, to allow the binding of the endothelial cells to the surface of the device. After several hours or days of culture, the device may be implanted into the host. Alternatively, the endothelial cells may be mixed with blood prior to application onto the implantation device.

More specifically, the number of cells deposited on the device coated with the peptides of the invention may be between about 10³ cells/cm² and 10¹² cells/cm² of device surface, typically about 5×10⁵ cells/cm². The cells are deposited in any convenient sterile medium, e.g. phosphate buffered saline (PBS), normal saline, M199, Dulbecco's Modified Eagles Medium (DMEM), and the like. The volume of medium will be sufficient to resuspend the cells, generally ranging from about 1 to 25 ml of medium.

After deposition, the device may be implanted immediately into the recipient or may be maintained in a conventional endothelial cell culture for a period of time. Cells employed for seeding on the implantable device may be obtained by any method known in the art. Cells may be obtained at the time of the implantation procedure using standard biopsy techniques, whether the procedure is angioplasty, open field surgery or for diagnostic purposes. The cells may also be dissociated with collagenase or trypsin and seeded directly into a matrix as exemplified below for immediate implantation or for culturing in vitro as required to generate the number of cells to be implanted. Specifically, cells may be isolated by standard methods described in, for example, Gimbrone, M. (1976) Progress Hemostasis and Thrombosis 3:1-28 and U.S. Pat. No. 5,131,907.

### Gene Therapy

Recently, the feasibility of gene therapy for modulating angiogenesis has been demonstrated (Takeshita, et al., Laboratory Investigation, 75:487-502 (1996); Isner, et al., Lancet, 348:370 (1996); U.S. Ser. No. 08/545,998; Tsurumi et al. (1996) Circulation 94(12):3281-90). The peptides of the invention find use in gene therapy for modification of vascular responses including restoration of endothelial integrity, repairing of ischemic injury, promoting angiogenesis, and the like. The peptides of the invention optimize cell delivery and cell retention to the site of interest, particularly OECs at the site of vascular injury.

The peptides of the invention can be expressed from vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

In one embodiment, a host cell is genetically modified to contain a stably integrated gene that confers a therapeutic effect by methods available in the art. In one embodiment, the gene that confers a therapeutic effect is a gene that encodes one or more of the peptides of the invention. Expression of this gene in an area in need of re-endothelialization and/or vascular repair can lead to the recruitment and retention of OECs at the site of repair.

In another embodiment, the gene that confers a therapeutic effect is a gene that encodes a therapeutic peptide or protein other than the peptides of the invention. When used in combination with the peptides of the invention, vascular repair is enhanced. For example, genetically-modified OECs or other suitable endothelial precursor cells can be used to administer therapeutic agents such as angiogenic enzymes, peptides and/or proteins with angiogenic activity, or nucleic acids or genes that encode therapeutic polypeptides involved in vascular repair. Nucleic acids encoding such therapeutic agents are introduced into OECs or endothelial precursor cells based upon their ability to optimally treat one or more vascular conditions. For example, the endothelial precursor cell can be designed to help control, diminish or otherwise facilitate improved arterial blood flow in the region of an atherosclerotic lesion.

Recombinant expression vectors are made and introduced into the cells using standard techniques, e.g., electroporation, lipid-mediated transfection, or calcium-phosphate mediated transfection, and cells containing stably integrated expression constructs are selected or otherwise identified, also using standard techniques known in the art. Methods for making recombinant DNA expression constructs, introducing them into eukaryotic cells, and identifying cells in which the expression construct is stably integrated and efficiently expressed, are described, for example, in Sambrook, et al., Molecular Cloning: A Laboratory Manual, 2d Edition, Cold Spring Harbor Laboratory Press (1989); Sambrook, et al., Molecular Cloning: A Laboratory Manual, 2d Edition, Cold Spring Harbor Laboratory Press (2001). Such methods useful for practicing the present invention are also described, for example, in U.S. Pat. No. 5,980,887.

The therapeutic agent nucleic acid sequences may be inserted into the vector by a variety of procedures. In general, DNA is inserted into an appropriate restriction endonuclease site(s) using techniques known in the art. See generally, Sambrook et al., 1989, Molecular Cloning, A Laboratory Manual, 2d ed., Cold Spring Harbor Laboratory Press, Cold Spring Harbor, N.Y.; Sambrook and Russell, Molecular Cloning: A Laboratory Manual, 3rd edition (Jan. 15, 2001) Cold Spring Harbor Laboratory Press, ISBN: 0879695765; Ausubel et al., Current Protocols in Molecular Biology, Green Publishing Associates and Wiley Interscience, NY (1989)). Construction of suitable expression vectors containing a therapeutic agent can employ standard ligation techniques that are known to the skilled artisan.

The expression cassette or vector of the invention includes a promoter. A promoter is a nucleotide sequence that controls expression of an operably linked nucleic acid sequence by providing a recognition site for RNA polymerase, and possibly other factors, required for proper transcription. A promoter includes a minimal promoter, consisting only of all basal elements needed for transcription initiation, such as a TATA-box and/or other sequences that serve to specify the site of transcription initiation. Any promoter able to direct transcription of an RNA encoding the selected therapeutic agent may be used. Accordingly, many promoters may be included within the expression cassette or vector of the invention. Some useful promoters include, constitutive promoters, inducible promoters, regulated promoters, cell specific promoters, viral promoters, and synthetic promoters. A promoter may be obtained from a variety of different sources. For example, a promoter may be derived entirely from a native gene, be composed of different elements derived from different promoters found in nature, or be composed of nucleic acid sequences that are entirely synthetic. A promoter may be derived from many different types of organisms and tailored for use within a given cell, for example, an OEC or other endothelial precursor cell.

Many mammalian promoters are known in the art that may be used in conjunction with the expression cassette of the invention. Mammalian promoters often have a transcription initiating region, which is usually placed proximal to the 5′ end of the coding sequence, and a TATA box, usually located 25 30 base pairs (bp) upstream of the transcription initiation site. The TATA box is thought to direct RNA polymerase II to begin RNA synthesis at the correct site. A mammalian promoter may also contain an upstream promoter element, usually located within 100 to 200 by upstream of the TATA box. An upstream promoter element determines the rate at which transcription is initiated and can act in either orientation (Sambrook et al., “Expression of Cloned Genes in Mammalian Cells”, in: Molecular Cloning: A Laboratory Manual, 2nd ed., 1989).

Mammalian viral genes are often highly expressed and have a broad host range; therefore sequences encoding mammalian viral genes often provide useful promoter sequences. Examples include the SV40 early promoter, mouse mammary tumor virus LTR promoter, adenovirus major late promoter (Ad MLP), and herpes simplex virus promoter. In addition, sequences derived from non-viral genes, such as the murine metallothionein gene, also provide useful promoter sequences. Expression may be either constitutive or regulated.

A mammalian promoter may also be associated with an enhancer. The presence of an enhancer will usually increase transcription from an associated promoter. An enhancer is a regulatory DNA sequence that can stimulate transcription up to 1000-fold when linked to homologous or heterologous promoters, with synthesis beginning at the normal RNA start site. Enhancers are active when they are placed upstream or downstream from the transcription initiation site, in either normal or flipped orientation, or at a distance of more than 1000 nucleotides from the promoter. (Maniatis et al., Science, 236:1237 (1987); Alberts et al., Molecular Biology of the Cell, 2nd ed., 1989)). Enhancer elements derived from viruses are often times useful, because they usually have a broad host range. Examples include the SV40 early gene enhancer (Dijkema et al., EMBO J., 4:761 (1985) and the enhancer/promoters derived from the long terminal repeat (LTR) of the Rous Sarcoma Virus (Gorman et al., Proc. Natl. Acad. Sci. USA, 79:6777 (1982b)) and from human cytomegalovirus (Boshart et al., Cell, 41: 521 (1985)). Additionally, some enhancers are regulatable and become active only in the presence of an inducer, such as a hormone or metal ion (Sassone-Corsi and Borelli, Trends Genet., 2:215 (1986); Maniatis et al., Science, 236:1237 (1987)).

It is understood that many promoters and associated regulatory elements may be used within the expression cassette of the invention to transcribe an encoded protein or peptide. The promoters described above are provided merely as examples and are not to be considered as a complete list of promoters that are included within the scope of the invention.

The expression cassettes and vectors of the invention may contain a nucleic acid sequence for increasing the translation efficiency of an mRNA encoding a therapeutic agent of the invention. Such increased translation serves to increase production of the therapeutic agent. Because eukaryotic mRNA does not contain a Shine-Dalgamo sequence, the selection of the translational start codon is usually determined by its proximity to the cap at the 5′ end of an mRNA. However, the nucleotides immediately surrounding the start codon in eukaryotic mRNA influence the efficiency of translation. Accordingly, one skilled in the art can determine what nucleic acid sequences will increase translation of a protein or peptide encoded by the expression cassettes and vectors of the invention.

Termination sequences can also be included in the cassettes and vectors of the invention. Usually, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-transcriptional cleavage and polyadenylation (Birmstiel et al., Cell, 41:349 (1985); Proudfoot and Whitelaw, “Termination and 3′ end processing of eukaryotic RNA”, in: Transcription and Splicing (eds. B. D. Hames and D. M. Glover) 1988; Proudfoot, Trends Biochem. Sci., 14:105 (1989)). These sequences direct the transcription of an mRNA that can be translated into the polypeptide encoded by the DNA. Examples of transcription terminator/polyadenylation signals include those derived from SV40 (Sambrook et al., “Expression of cloned genes in cultured mammalian cells”, in: Molecular Cloning: A Laboratory Manual, 1989).

As indicated above, nucleic acids encoding the therapeutic agents can be inserted into any convenient vector. Vectors that may be used include, but are not limited to, those that can be replicated in prokaryotes and eukaryotes. For example, vectors may be used that are replicated in bacteria, yeast, insect cells, and mammalian cells. Examples of vectors include plasmids, phagemids, bacteriophages, viruses, retroviruses, cosmids, and F-factors. However, specific vectors may be used for specific cells types. Additionally, shuttle vectors may be used for cloning and replication in more than one cell type. Such shuttle vectors are known in the art. The nucleic acid constructs or libraries may be carried extrachromosomally within a host cell or may be integrated into a host cell chromosome. Numerous examples of vectors are known in the art and are commercially available. (Sambrook and Russell, Molecular Cloning: A Laboratory Manual, 3rd edition (Jan. 15, 2001) Cold Spring Harbor Laboratory Press, ISBN: 0879695765; New England Biolab, Beverly, Mass.; Stratagene, La Jolla, Calif.; Promega, Madison, Wis.; ATCC, Rockville, Md.; CLONTECH, Palo Alto, Calif.; Invitrogen, Carlabad, Calif.; Origene, Rockville, Md.; Sigma, St. Louis, Mo.; Pharmacia, Peapack, N.J.; USB, Cleveland, Ohio). These vectors also provide many promoters and other regulatory elements that those of skill in the art may include within the nucleic acid constructs of the invention through use of known recombinant techniques.

Recombinant retroviruses can also be used which are constructed to carry or express at least one selected peptide of interest. Retrovirus vectors that can be employed include those described in EP 0 415 731; WO 90/07936; WO 94/03622; WO 93/25698; WO 93/25234; U.S. Pat. No. 5,219,740; WO 93/11230; WO 93/10218; Vile and Hart, Cancer Res. 53:3860-3864 (1993); Vile and Hart, Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. No. 4,777,127; GB Patent No. 2,200,651; WO 91/02805; and EP 0 345 242.

Packaging cell lines suitable for use with the above-described retroviral vector constructs may be readily prepared (see PCT publications WO 95/30763 and WO 92/05266), and used to create producer cell lines (also termed vector cell lines) for the production of recombinant vector particles.

It is recognized that alphavirus-based vectors can be used that can function as gene delivery vehicles. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532). Representative examples of such vector systems include those described in U.S. Pat. Nos. 5,091,309; 5,217,879; and 5,185,440; and PCT Publication Nos. WO 92/10578; WO 94/21792; WO 95/27069; WO 95/27044; and WO 95/07994.

Gene delivery vehicles of the present invention can also employ parvovirus such as adeno-associated virus (AAV) vectors. Representative examples include the AAV vectors disclosed by Srivastava in WO 93/09239, Samulski et al., J. Vir. 63:3822-3828 (1989); Mendelson et al., Virol. 166:154-165 (1988); and Flotte et al., P.N.A.S. 90:10613-10617 (1993).

Representative examples of adenoviral vectors include those described by Berkner, Biotechniques 6:616-627 (Biotechniques); Rosenfeld et al., Science 252:431-434 (1991); WO 93/19191; Kolls et al., P.N.A.S.:215-219 (1994); Kass-Eisler et al., P.N.A.S. 90:11498-11502 (1993); Guzman et al., Circulation 88:2838-2848 (1993); Guzman et al., Cir. Res. 73:1202-1207 (1993); Zabner et al., Cell 75:207-216 (1993); Li et al., Hum. Gene Ther. 4:403-409 (1993); Cailaud et al., Eur. J. Neurosci. 5:1287-1291 (1993); Vincent et al., Nat. Genet. 5:130-134 (1993); Jaffe et al., Nat. Genet. 1:372-378 (1992); and Levrero et al., Gene 101:195-202 (1992). Exemplary adenoviral gene therapy vectors employable in this invention also include those described in WO 94/12649, WO 93/03769; WO 93/19191; WO 94/28938; WO 95/11984 and WO 95/00655. Administration of DNA linked to killed adenovirus as described in Curiel, Hum. Gene Ther. 3:147-154 (1992), may be employed.

A nucleic acid construct, or an expression vector can also be inserted into any mammalian vector that is known in the art or that is commercially available, for example, as provided by CLONTECH (Carlsbad, Calif.), Promega (Madision, Wis.), or Invitrogen (Carlsbad, Calif.). Such vectors may contain additional elements such as enhancers and introns having functional splice donor and acceptor sites. Nucleic acid constructs may be maintained extrachromosomally or may integrate in the chromosomal DNA of a host cell. Mammalian vectors include those derived from animal viruses, which require trans-acting factors to replicate. For example, vectors containing the replication systems of papovaviruses, such as SV40 (Gluzman, Cell, 23:175 (1981)) or polyomaviruses, replicate to extremely high copy number in the presence of the appropriate viral T antigen. Additional examples of mammalian vectors include those derived from bovine papillomavirus and Epstein-Barr virus. Additionally, the vector may have two replication systems, thus allowing it to be maintained, for example, in mammalian cells for expression and in a prokaryotic host for cloning and amplification. Examples of such mammalian-bacteria shuttle vectors include pMT2 (Kaufman et al., Mol. Cell. Biol., 9:946 (1989)) and pHEBO (Shimizu et al., Mol. Cell. Biol., 6:1074 (1986)).

Methods for introduction of heterologous polynucleotides into mammalian cells are known in the art and include lipid-mediated transfection, dextran-mediated transfection, calcium phosphate precipitation, polybrene-mediated transfection, protoplast fusion, electroporation, encapsulation of -the polynucleotide(s) in liposomes, biollistics, and direct microinjection of the DNA into nuclei. The choice of method depends on the cell being transformed as certain transformation methods are more efficient with one type of cell than another. (Felgner et al., Proc. Natl. Acad. Sci., 84:7413 (1987); Felgner et al., J. Biol. Chem., 269:2550 (1994); Graham and van der Eb, Virology, 52:456 (1973); Vaheri and Pagano, Virology, 27:434 (1965); Neuman et al., EMBO J., 1:841 (1982); Zimmerman, Biochem. Biophys. Acta., 694:227 (1982); Sanford et al., Methods Enzymol., 217:483 (1993); Kawai and Nishizawa, Mol. Cell. Biol, 4:1172 (1984); Chaney et al., Somat. Cell Mol. Genet., 12:237 (1986); Aubin et al., Methods Mol. Biol., 62:319 (1997)). In addition, many commercial kits and reagents for transfection of eukaryotic cells are available.

Following transformation or transfection of a nucleic acid into a cell, the cell may be selected for the presence of the nucleic acid through use of a selectable marker. A selectable marker is generally encoded on the nucleic acid being introduced into the recipient cell. However, co-transfection of selectable marker can also be used during introduction of nucleic acid into a host cell. Selectable markers that can be expressed in the recipient host cell may include, but are not limited to, genes that render the recipient host cell resistant to drugs such as actinomycin C₁, actinomycin D, amphotericin, ampicillin, bleomycin, carbenicillin, chloramphenicol, geneticin, gentamycin, hygromycin B, kanamycin monosulfate, methotrexate, mitomycin C, neomycin B sulfate, novobiocin sodium salt, penicillin G sodium salt, puromycin dihydrochloride, rifampicin, streptomycin sulfate, tetracycline hydrochloride, and erythromycin. (Davies et al., Ann. Rev. Microbiol., 32: 469 (1978)). Selectable markers may also include biosynthetic genes, such as those in the histidine, tryptophan, and leucine biosynthetic pathways. Upon transfection or transformation of a cell, the cell is placed into contact with an appropriate selection agent.

The expression cassettes may further comprise a selectable suicide gene, such as thymidine kinase (TK), which allows negative selection of grafted cells upon completion of treatment or in the event of undesired complications. TK-expressing cells can be negatively selected by the administration of gancyclovir according to methodology known in the art. Alternatively, the cassette may encode cytosine deaminase, which causes the cells to die in the presence of added 5-fluorocytosine. The expressed gene can be lethal as a toxin or lytic agent.

It is recognized that the OEC may be isolated and modified by genetic modification prior to delivery to a site of interest. See, for example, Nabel et al. 1989 Science 244:1342-1344; Wilson et al. 1989 Science 244:1344-1346; Iwaguro et al. 2002 Circulation 105:732-738; Jevrumovic et al. 2004 Am J Physiol Heart Circ Physiol 287:H494-500; all of which are herein incorporated by reference.

### Laboratory and Clinical Uses

The invention also encompasses methods for the identification and isolation of additional peptides capable of specifically binding OEC. The peptides can be isolated by the methods set forth herein. Phage display technology is well-known in the art and can be used to identify candidate peptides from a library of diverse peptides. Phage display describes a selection technique in which a library of variants of a peptide or protein is expressed on the outside of a phage virion, while the genetic material encoding each variant resides on the inside (Sidhu et al. (2003) Chembiochem, 4:14-25; Ferrer et al. (1999) J. Pept. Res., 54:32-42; and, BouHamdan et al. (1998) J. Biol. Chem. 273:8009-8016). This creates a physical linkage between each variant protein sequence and the DNA encoding it, which allows rapid partitioning based on binding affinity to a given target molecule (antibodies, enzymes, cell-surface receptors, etc.) by an in vitro selection process called “panning” or “biopanning” (Whaley et al. (2000) Nature 405:665-668). Panning methods can include, for example, solution phase screening, solid phase screening, or cell-based screening.

In its simplest form, panning is carried out by incubating a library of phage-displayed peptides with a plate (or bead) coated with the target, washing away the unbound phage, and eluting the specifically bound phage. The eluted phage is then amplified and taken through additional binding/amplification cycles to enrich the pool in favor of binding sequences. After 3-4 rounds, individual clones are characterized by DNA sequencing and ELISA. Once a candidate peptide is identified, directed or random mutagenesis of the sequence may be used to optimize the binding properties of the peptide.

In another embodiment, antibodies can be raised against the peptides of the invention. These antibodies can be used to isolate or identify OECs by contacting the antibody with a population of cells that has been incubated with a sufficient amount of one or more of the OEC-binding peptides disclosed herein. The antibodies can be free in solution or bound to a solid support as discussed infra. Methods for producing antibodies are well known in the art (see, for example, Harlow and Lane (1988) “Antibodies: A Laboratory Manual,” Cold Spring Harbor Laboratory, Cold Spring Harbor, N.Y.; U.S. Pat. No. 4,196,265).

OECs can be also be identified and/or purified by contacting the cells with the OEC-binding peptides of the invention. The specific and selective binding of the OECs to the peptide(s) permits the OECs to be sufficiently distinguished from contaminating cells that do not express the peptide-binding antigen. The term purified as applied to the endothelial precursor cell population utilized herein means that the population is significantly enriched in endothelial precursor cells relative to the crude population of cells from which the endothelial precursor cells are isolated. The peptides can be part of one or more reagents or kits suitable for these purposes.

When used for isolating and/or characterizing the populations of OECs, the peptides of the invention can be conjugated with labels that expedite identification and separation of the OECs from other cells in a population or sample. Examples of such labels include magnetic beads, biotin, which may be removed by avidin or streptavidin, fluorochromes, which may be used in connection with a fluorescence-activated cell sorter, and the like.

In one embodiment, the peptide is attached to a solid support. Some suitable solid supports include nitrocellulose, agarose beads, polystyrene beads, hollow fiber membranes, and plastic petri dishes. For example, the molecule can be covalently linked to Pharmacia Sepharose 6 MB macro beads. The exact conditions and duration of incubation for the solid phase-linked peptides with the crude cell mixture will depend upon several factors specific to the system employed, as is well known in the art. Cells that are bound to the peptide are removed from the cell suspension by physically separating the solid support from the cell suspension. For example, the unbound cells may be eluted or washed away with physiologic buffer after allowing sufficient time for the solid support to bind the OECs.

The bound cells are separated from the solid phase by any appropriate method, depending mainly upon the nature of the solid phase. For example, bound cells can be eluted from a plastic petri dish by vigorous agitation. Alternatively, bound cells can be eluted by enzymatically “nicking” or digesting an enzyme-sensitive “spacer” sequence between the solid phase and the peptide (or an antibody raised against the peptide as discussed supra). Suitable spacer sequences bound to agarose beads are commercially available, for example, from Pharmacia.

The eluted, enriched fraction of cells may then be washed with a buffer by centrifugation and preserved in a viable state at low temperatures for later use according to conventional technology. The cells may also be used immediately, for example by being infused intravenously into a recipient.

The peptides disclosed herein may also be used to identify and/or purify OECs by means of flow cytometry, for example by means of a fluorescence-activated cell sorter (FACS), such as those manufactured by Becton-Dickinson under the names FACScan or FACSCalibur. By means of this technique, OECs are tagged with a particular fluorescent dye (i.e., “stained”) by means of one or more peptides of the invention which have been conjugated to such a dye. When the stained cells are placed on the instrument, a stream of cells is directed through an argon laser beam that excites the fluorochrome to emit light. This emitted light is detected by a photo-multiplier tube (PMT) specific for the emission wavelength of the fluorochome by virtue of a set of optical filters. The signal detected by the PMT is amplified in its own channel and displayed by a computer in a variety of different forms—e.g., a histogram, dot display, or contour display. Thus, fluorescent cells which emit at one wavelength express a molecule that is reactive with the specific fluorochrome-labeled peptide, whereas non-fluorescent cells do not express this molecule. The flow cytometer is also semi-quantitative in that it displays the amount of fluorescence (fluorescence intensity) expressed by the cell. This correlates, in a relative sense, to the number of the peptide-binding molecules expressed by the cell.

Fluorochromes which are typically used with FACS machines include fluorescein isothiocyanate (FITC), which has an emission peak at 525 nm (green), R-phycoerythrin (PE), which has an emission peak at 575 nm (orange-red), propidium iodide (PI), which has an emission peak at 620 nm (red), 7-aminoactinomycin D (7-AAD), which has an emission peak at 660 nm (red), R-phycoerythrin Cy5 (RPE-Cy5), which has an emission peak at 670 nm (red), and allophycocyanin (APC), which has an emission peak at 655-750 nm (deep red).

These and other types of FACS machines may have the additional capability to physically separate the various fractions by deflecting the cells of different properties into different containers.

In another embodiment, OECs are concentrated (or “enriched”) from blood or blood products. In this manner, blood is withdrawn directly from the circulating peripheral blood of a donor and percolated continuously through a column containing the solid phase-linked binding molecule, such as an OEC-binding peptide, to capture OECs. The OEC-depleted blood is returned immediately to the donor's circulatory system by methods known in the art, such as hemapheresis. The blood is processed in this way until a sufficient number of progenitor cells binds to the column. The stem cells are then isolated from the column by methods known in the art. This method allows rare OECs to be harvested from a very large volume of blood. Transplantation of new cells into the damaged blood vessels has the potential to repair damaged vascular tissue, e.g., veins, arteries, capillaries, thereby restoring vascular function.

The following examples are offered by way of illustration and not by way of limitation.

### EXPERIMENTAL

### Example 1

### Introduction

Progenitor cell-based regenerative strategies offer new perspectives in cell therapies and tissue engineering for achieving an effective revascularization of ischemic or injured tissues. Asahara et al. (1997) first described that peripheral blood contains a small subset of circulating bone marrow derived cells termed endothelial progenitor cells (EPC). Recent reports from in-vitro studies underline the observation that so-called EPC represent a heterogeneous population of cells with different capacities to assume a differentiated and functional endothelium phenotype in-vitro or with respect to proliferation (Gulati et al. 2003a; Gulati and Simari 2004; Rehman et al. 2003). Cultures from peripheral blood contain cells termed early-EPC that share some endothelial but also monocytic characteristics and exhibit a restricted capacity of expansion. Another cell population isolated from peripheral blood cultures is the so called late-EPC or blood outgrowth endothelial cells (BOEC) that have a cobblestone morphology characteristic of an endothelial phenotype. Furthermore, these cells express several endothelial markers and have high proliferative capacity (Lin et al. 2000).

Endothelial progenitors have been implicated in myocardial repair after infarction, in the propagation of angiogenesis following ischemia, and in vascular repair after injury (Gulati et al. 2003b; Hajitou et al. 2006; Iwakura et al. 2003). In spite of the enormous therapeutic potential of these cells, the molecular characteristics and EPC biology are incompletely understood. Endothelial progenitor cell-specific markers are needed to facilitate the development of future progenitor therapies which may be either pharmacological or device based. Towards this end, phage display technology was used to identify new peptide ligands that bind with high affinity and specificity to human blood outgrowth endothelial cells (HBOEC).

Since the invention of phage display systems in 1985 (Smith 1985), display technologies have proven to be a valuable tool for a variety of biological, clinical and biotechnological applications (Ballard et al. 2006a; Ballard et al. 2006b; Edelberg et al. 2004; Smith and Petrenko 1997). These include the characterization of receptor- and antibody-binding sites, the study of protein ligand interactions, and the isolation and evolution of proteins or enzymes exhibiting improved or otherwise altered binding characteristics with their ligands. Phage display screening relies on the use of chimeric proteins that consist of a target sequence fused to a phage coat protein to achieve display. Using standard molecular biology techniques, the DNA sequence of the inserted region can be randomized to create a library of phage, each of which will synthesize a different version of the chimera on its surface. By incubating the library with a target of interest, washing out weak or non binders, and repeating the process to enrich for tight binders, a subset can be selected from the original library exhibiting the ability to tightly interact with the desired target. This in vitro selection process is known as biopanning. Because the chimera is encoded within the phage genome the identity of the selected sequences, e.g., their amino acid composition can be deduced by DNA sequencing.

Biopanning on whole cells with no directive pressures on the selective scheme has many advantages: the receptors are more likely to be in their native conformation, with all their natural posttranslational modifications, and neither purification nor prior knowledge of a particular target receptor is required. An additional strength of this approach is that it is highly inductive, in that it does not rely on knowledge of which cell surface molecules are present, their concentration, or their specificity. Furthermore, this approach ensures that the selected peptide sequence binds to its target in the presence of many other biological macromolecules and allows for selection of membrane proteins that are often difficult to express and purify.

### Materials and Methods

Isolation of Human Blood Outgrowth Endothelial Cells from Peripheral Blood:

The use of human material described in this study was approved by the responsible ethical committee. Fresh blood was collected from healthy volunteer donors by venipuncture and anticoagulated with buffered sodium citrate. The anticoagulated blood was diluted 1:1 with HBSS (Sigma-Aldrich) containing 1 mM EDTA and 0.5% BSA. Buffy coat mononuclear cells were obtained from diluted blood by density gradient centrifugation method using Histopaque 1077 (Sigma-Aldrich) (Lin et al. 2000). The cells were washed in PBS three times at 400 g for 10 min before culturing. Buffy coat mononuclear cells from 100 ml peripheral blood were resuspended in EGM-2 medium (endothelial cell growth medium 2; Cambrex Bioscience, Walkersville, Md.) without further subpopulation enrichment procedures and placed into one well of a six well plate coated with type 1 collagen (BD Biosciences, Bedford, Mass.). The plate was incubated at 37° C. in a humidified environment with 5% CO₂. Non adherent cells were removed after 48 hours and every second day thereafter. Colonies with cobblestone morphology appeared after 3-4 weeks in culture. These cells were cultured until they formed larger colonies. Colonies were selected, trypsinized, and expanded over several passages by using standard cell culture procedures.

### Isolation of Human Lymphocytes and Neutrophils:

Lymphocytes and neutrophils were obtained from the same preparation. Lymphocytes were collected from the supernatant of the cultured buffy coat mononuclear cells that were allowed to adhere for 48 hours. The cell concentration was adjusted to 1.10⁵ cells per ml.

Neutrophils were collected from the lower portion of the density gradient preparation. The upper layers were removed and processed as described above. The neutrophil-rich Histopaque layer was transferred to a centrifuge tube and diluted in fresh RPMI to wash the cells free of Histopaque. The suspension was centrifuged at 700×g for 15 min at room temperature. The supernatant was aspirated and the pellet resuspended in 10 ml of RPMI and centrifuged at 700×g for 10 min at room temperature. Contaminating red cells were lysed by quickly re-suspending the pellet in sterile water at 4° C. After 30 sec an equal volume of 1.8% saline solution was added in order to return the solution to isotonicity. The suspension was then centrifuged for 10 min at 250×g at 4° C. The cell lysis step was repeated. The final pellet was re-suspended and concentration adjusted to 1.10⁵ cells per ml.

### Human Umbilical Vein Endothelial Cells (HUVEC):

HUVEC were from the American Type Culture Collection (ATCC, Manassas, Va.). Passages 4 to 8 were used in this study. HUVEC were cultured in EGM-MV medium (Cambrex) at 37° C. in an incubator with humid atmosphere and 5% CO₂.

Peripheral blood human HL-60 promyelocytic cells from the American Type Culture Collection (ATCC) were cultured in RPMI 1640 medium supplemented with 20% fetal bovine serum.

### Biopanning Procedure

Cells at 80% confluence were detached by treating with 0.05% trypsin-EDTA, washed once with EGM-2 medium and resuspended in EGM-2 containing 1% BSA at 1.10⁵ cells per ml. In the pre-clearing step, 1 ml of HUVEC suspension at 1.10⁵ cells per ml were incubated with 10 μl of PhD-12 peptide phage display system (New England Biolabs, Beverly, Mass.) within 1.5 ml Eppendorf tube for 2 hours at 4° C.; the mixture was then centrifuged. In the screening step, the unbound phage pool remaining in the supernatant was transferred to a fresh tube and incubated with 1 ml of HBOEC at 1.10⁵ cells per ml. After 1 hour incubation at 4° C., the cell-phage complexes were separated by centrifugation. Following five intensive washes with TBS-0.5% Tween-20 buffer the bound phage was non-specifically eluted with 0.2 M Glycine-HCl buffer (pH 2.2) for 10 min. The eluate was immediately neutralized by 1M Tris.HCl buffer (pH 9.0). An aliquot of the eluted phage was used for determining titer by plaque assay. The rest of the phage eluate was amplified in mid-log phase E. coli ER2738 (New England Biolabs), and purified by precipitation with polyethylene glycol. An aliquot of the amplified phage was subsequently re-applied to newly trypsinized cells for a total of three biopanning rounds and two amplification steps.

### DNA Sequencing

After three rounds of biopanning E. Coli ER2738 were infected with the recovered phage and then plated onto LB agar plates. Single phage colonies were picked and amplified in LB medium. DNA was purified and sequenced by using a primer hybridizing to −96 position of the insert following the manufactures instructions. DNA sequencing was performed by the UNC-CH Genome Analysis Facility (Chapel Hill, N.C.).

### Homogeneous Phage Recovery

Once isolated individual phage clones were subjected to evaluation of relative binding. High titer stocks of homogeneous phage were generated. Serially diluted phage (1.10⁹ pfu, 1.10¹⁰ pfu, and 1.10¹¹ pfu) were incubated with HBOEC (1.10⁵ cells) for 1 h at 4° C. and then subjected to the same wash protocol used for the selection experiments. In parallel the same procedure was carried in a blocked Eppendorf tube without HBOEC to test for non specific binding for each selected sequence to the plastic container. Binding ratio is defined as recovery of phage bound to the target cells normalized to the recovery of phage non-specifically bound to the plastic.

### Assaying for Binding Specificity

The specificity of HBOEC-selected phage clones was determined by biopanning on a panel of other cell types. The biopanning procedure was carried as described above with the exception of including the pre-clearing incubation step.

### Immunofluorescence Staining:

Cells were seeded on glass cover slips coated with rat tail collagen in 12-well plates. Cells were incubated at 37° C. for 30 min prior to fixation with 10 mg/ml DiI-Ac-LDL (acetylated low density lipoprotein DiI complex; Molecular Probes). After fixation with 4% paraformaldehyde, cells were permiabilized with 0.1% Triton-X in PBS. Cells were then incubated with rabbit anti-human vWF antibody (von Willebrand Factor; DACO) in PBS-1% BSA, and then with secondary antibody coupled with AlexaFluor 488 (Molecular Probes). Cell nuclei were counter stained with DAPI (Sigma-Aldrich).

### Peptide Synthesis:

All active and control peptide sequences were synthesized using standard FMoc chemistry by solid phase peptide synthesizer (Commonwealth Biotech. Inc., Richmond, Va.). The peptides were purified by HPLC and chemical purity was confirmed by mass spectrometry (MALDITOF).

### Proliferation Assay:

Cell proliferation assays were performed in triplicate in 12-well culture plates. HBOEC (2000 cells per well) were plated and grown overnight in EGM-2 supplemented with 2% FBS. Cells were quiesced in EBM-2 medium containing 0.5% FBS for 16 h. Cells were re-fed with medium containing escalating concentrations of free peptides. Cell proliferation at 12 h, 36 h, and 96 h was determined by total and viable cell counts by Trypan blue exclusion. Proliferation was normalized to the cell number before the addition of free peptides.

### In Vitro Tube Formation Assay:

For the angiogenesis and cell migration assays, cells were detached with 1 mM EDTA (Sigma-Aldrich) to avoid cell membrane antigen proteolysis. After detachment, HBOEC were incubated with peptides in a dose dependent manner, seeded on Matrigel matrix in 96-well plate (10000 cells per well) and cultured for 6 hours at 37° C. with 5% CO2. Capillary-like structures were examined by phase contrast microscopy and digital images were taken and quantified by computer assisted analysis.

### Cell Migration Assay:

HBOEC migration was measured by using a 48-well Boyden chamber with 8 μm pore-size filters. EDTA-detached-HBOEC were incubated with peptides in a dose dependent manner and seeded at a density 5000 cells per well. Recombinant human VEGF (25 ng/ml, R&D systems) was diluted in EBM-2 medium supplemented with 2% FBS and placed in the lower chamber. Cells were incubated at 37° C. for 12 hours.

### Response to VEGF Assay:

Response to VEGF assays were performed in triplicate in 12-well culture plates. HBOEC (2000 cells per well) were plated and grown overnight in EGM-2 supplemented with 2% FBS. Cells were quiesced in EBM-2 medium containing 0.5% FBS for 16 h. Cells were re-fed with medium containing escalating concentrations of free peptides, VEGF (25 ng/ml) and 2% FBS in EBM-2 medium. Cell proliferation for 96 h and total and viable cell counts were determined by Trypan blue exclusion. Cell numbers were normalized to conditions without VEGF.

### Synthesis of Peptide Modified Terpolymers:

Peptide sequences were immobilized to methacrylic terpolymers via one step chain transfer controlled free radical polymerization as described by Fussell and Cooper (Fussell 2004a and Fussell 2004b). The monomers used in the reactions were n-hexyl methacrylate (HMA) (Alfa Aesar, Ward Hill, Mass.), methyl methacrylate (MMA) (ACROS Organics, Pittsburgh, Pa.), and methacrylic acid (MAA) (ACROS Organics, Pittsburgh, Pa.), with 2,2-azobisisobutyronitrile (AIBN) (Aldrich Chemical, Milwaukee, Wis.) as the initiator. The molar ratio of the monomers in the reaction mixture was HMA:MMA:MAA-20:78:2. The peptides were added with the monomers after the solvent was purged with argon. The reaction temperature for the polymerization was 55-60° C. and reactions were carried out overnight.

### Amino Acid Analysis:

The amount of peptide incorporation was determined from amino acid analysis performed by Commonwealth Biotechnologies, Inc. (Richmond, Va.).

### Cell Binding Assay:

Peptide grafted materials were coated on round microscope cover glass slides. Glass slides were sterilized by immersion in 70% ethanol. After washing with PBS, the cover slips were placed in tissue culture polystyrene plates and incubated with an HBOEC suspension in serum free medium at a concentration 1×104 cells/ml. After two hours of incubation, medium was aspirated, loosely attached cells were washed with PBS and the cells were fixed with 4% paraformaldehyde. Cell nuclei were stained with DAPI and examined under fluorescent microscope. Digital images were taken from 15 random fields per sample and quantified by computer assisted analysis.

### Data Analysis:

Data are representative of at least three independent experiments and quantitative analyses are presented as means±SD. Statistical analysis, where applicable, was performed in Microsoft Excel. A two-tailed unpaired Student's t-test was used to compare the differences. A value of P<0.05 was considered statistically significant.

### Results

Identification of Phage Clones that Bind HBOEC:

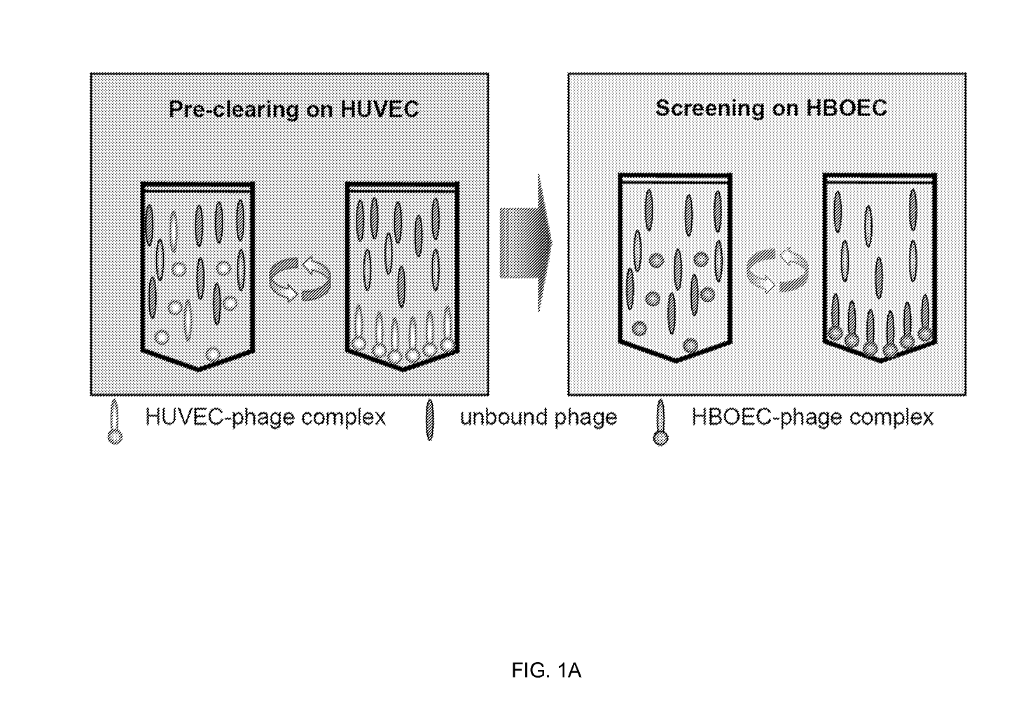

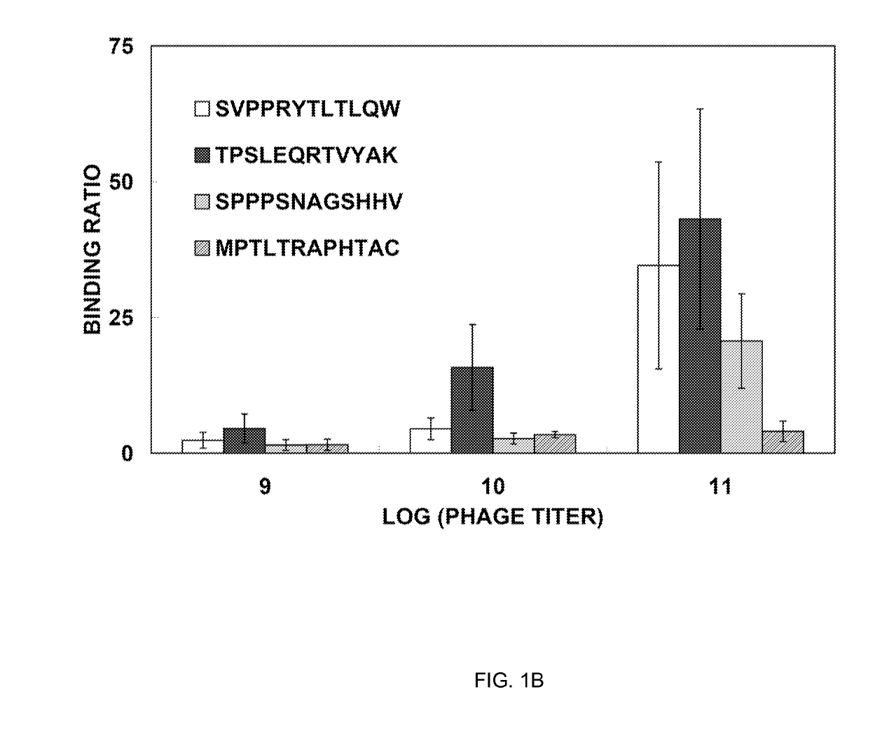

The strategy adopted for selection of peptide ligands that bind specifically to HBOEC involved a two-step biopanning procedure as outlined on FIG. 1A. First, to decrease non-specific binding the phage library was pre-cleared with non-HBOEC. The phage library was incubated with HUVEC and centrifuged to separate HUVEC-phage complexes and unbound phage clones. Second, the unbound phage pool was incubated with HBOEC for 1 hour at 4° C. After stringent washing, the phage that bound to HBOEC were harvested and amplified back to the original input titer of the library and used for subsequent rounds of biopanning. After three rounds of selection, individual phage were isolated and the peptide ligand sequences were determined for 40 randomly chosen phage clones. Thirty-eight different peptide sequences were deduced and two phage clones contained no inserts. The sequencing results are summarized in Table 1. The population of peptides contained a number of potential consensus motifs. By scoring the most commonly observed amino acids at each position, the primary consensus sequence, SPTPS(P/L)PPSAGG (SEQ ID NO:39), was determined. Although this individual peptide was not isolated in the screen, the consensus motifs TPS and PPS appeared in the isolated peptide ligands (see Table 1). Analysis of peptide sequences using BLAST (Altschul et al. 1997), identified a number of different homologies listed in Table 2.

### Homogeneous Phage Recovery

After selection of putative HBOEC specific ligands, pure high titer stocks of homogeneous phage were generated for further assessment of binding characteristics. The initial analysis, disclosed herein, includes four phage clones: SVPPRYTLTLQW (SEQ ID NO:7), TPSLEQRTVYAK (SEQ ID NO:17), SPPPSNAGSHHV (SEQ ID NO:32), and MPTLTRAPHTAC (SEQ ID NO:37) bearing different consensus motifs (see also Table 1). FIG. 1B, shows the concentration dependence of homogeneous recovery for the selected phage clones. It is seen from the figure that all four ligands bind HBOEC over a range of concentrations in a dose-dependent manner. The highest recovery is displayed by the phage expressing the TPSLEQRTVYAK peptide. This finding suggests either higher affinity for the TPSLEQRTVYAK ligand or availability of more binding sites on the HBOEC surface. The lowest affinity/avidity was exhibited by a ligand containing the MPT motif.

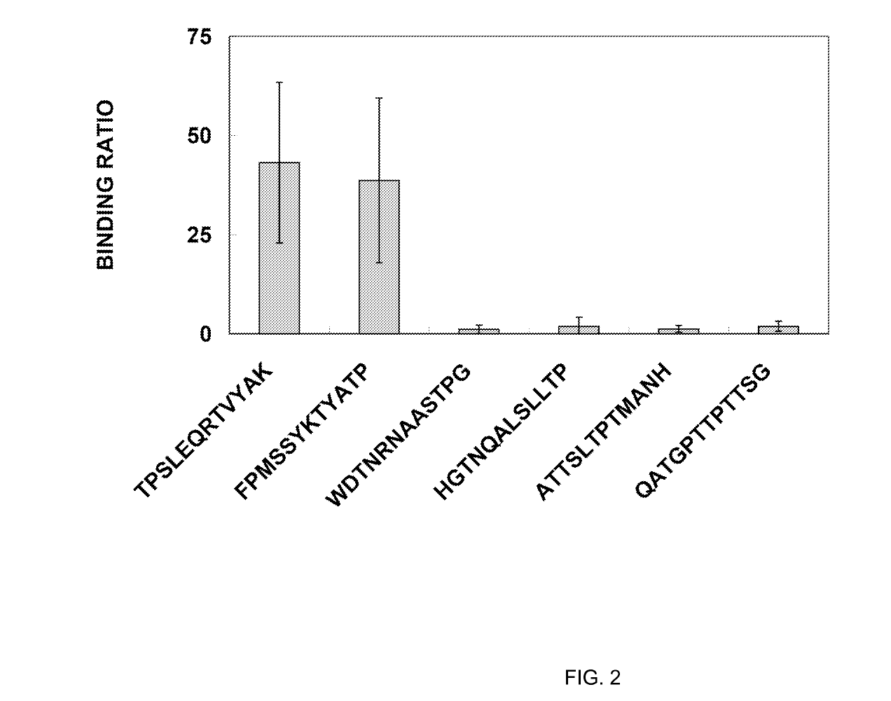

Next, the question of whether the consensus motif in a sequence determines ligand binding was addressed. Homogeneous recovery was examined in a group of phage bearing the TP(S/T/G) motif. HBOEC (1.10⁵ cells) were biopanned on 1.10¹¹ pfu of homogeneous phage. FIG. 2 shows different recoveries. Although some phage demonstrate lower recovery and therefore lower affinity/avidity for HBOEC, other phage display higher recoveries. Again the phage expressing the TPSLEQRTVYAK peptide ligand showed the highest recovery, having a binding ratio of 43±20. The binding profile on FIG. 2 demonstrates that recovery may not be determined solely by the consensus motif and that the amino acids flanking the consensus motif may impact binding interactions as well.

### Cell Specificity

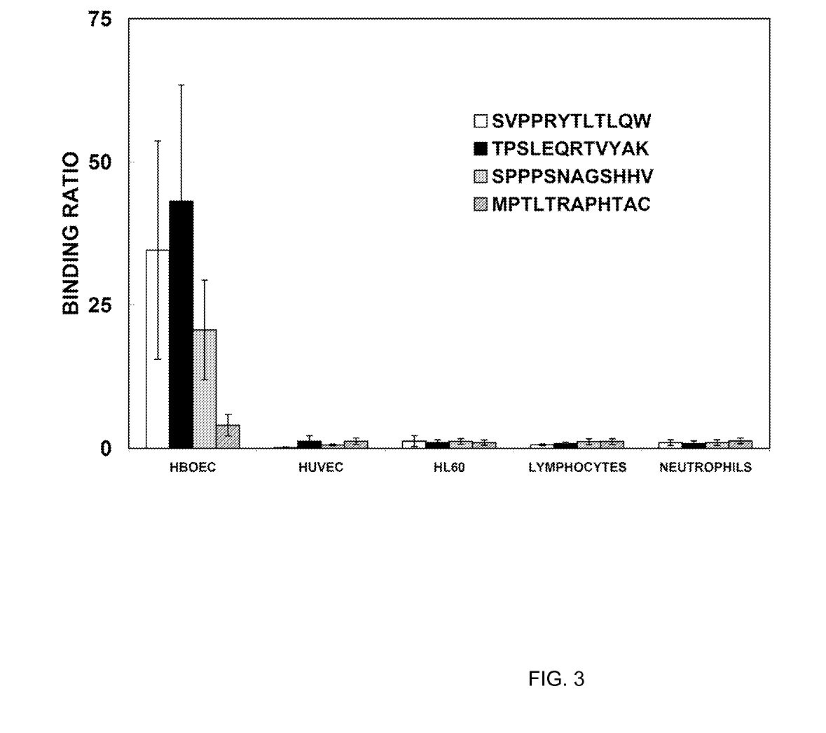

While phage clones expressing the SVPPR YTLTLQW, TPS LEQRTVYAK, SPPPS NAGSHHV, and MPT LTRAPHTAC ligands demonstrate good recoveries, there is no assurance that the selected sequences will exhibit specificity for the target cells. To assess cell specificity, the above phage clones were screened against a panel of other cell types. FIG. 3 shows the specificity profile of the selected phage clones. No significant recovery is seen for any of the clones for the cell types tested except for the target cells.

### Functional Characterization of Phage Display Selected Ligands on a Peptide Level.

To examine the effect of phage display-selected peptide ligands on endothelial cell function, free peptides were synthesized by a solid phase peptide technology. The C-terminus of the active peptides was extended with a Gly-Gly-Gly-Ser linker (SEQ ID NO:47) followed by an additional cysteine.

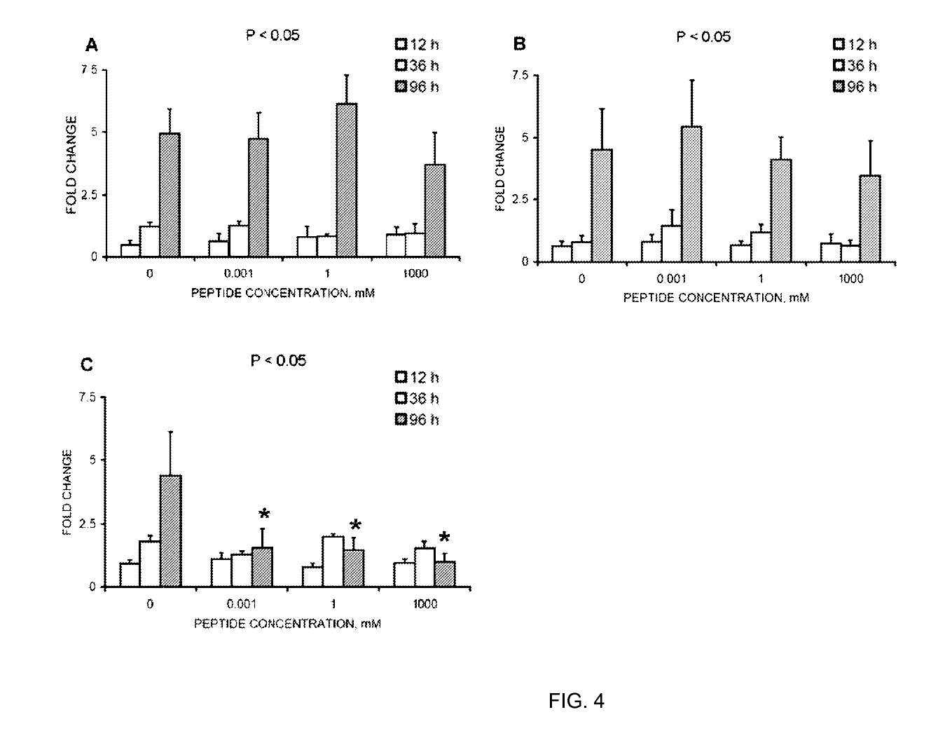

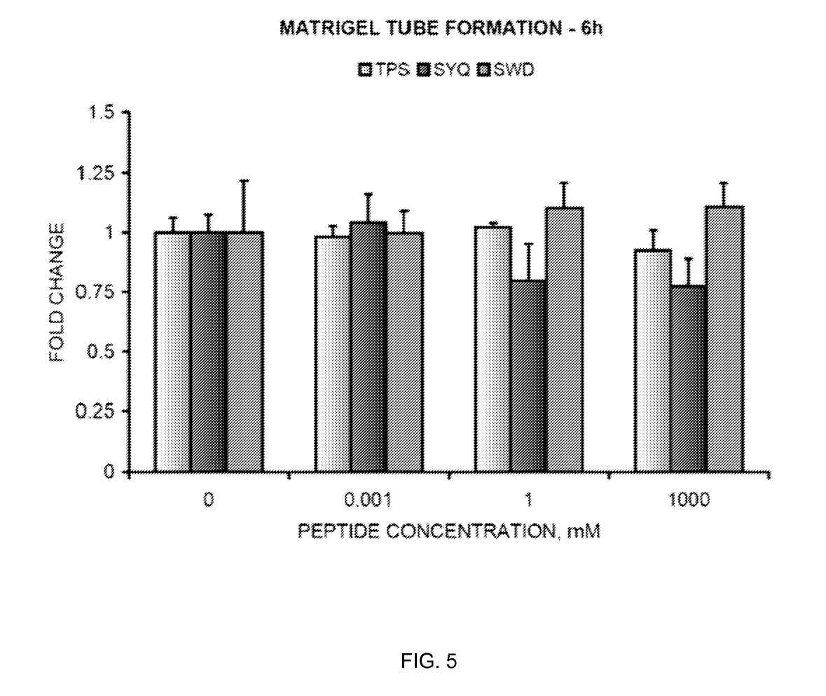

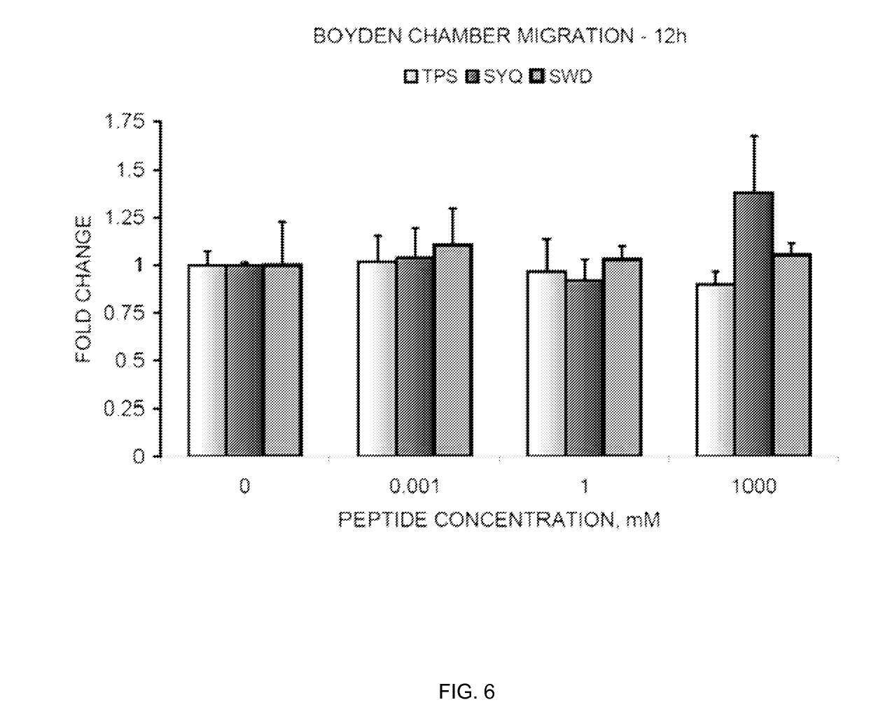

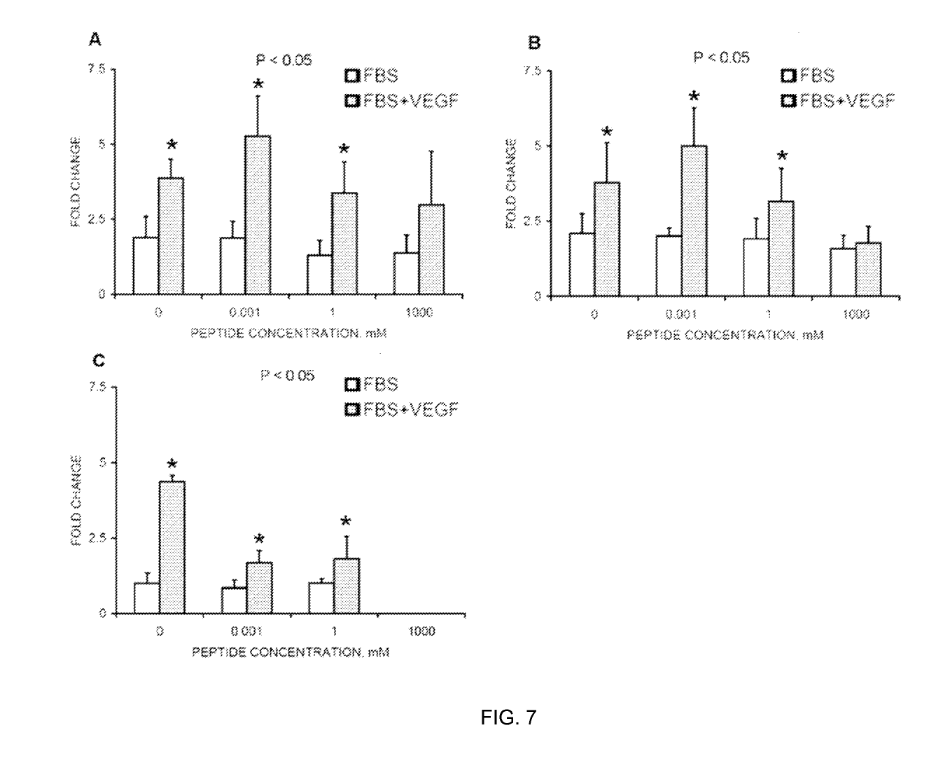

Proliferation data of HBOEC incubated with increasing concentrations of free peptides are presented in FIG. 4. The results from the tube formation, migration, and response to VEGF assays are shown in FIGS. 5, 6, and 7 respectively. As can be seen from these figures, the TPS- and SYQ-peptides supported endothelial cell function while the SWD-peptide displayed apoptotic properties and caused cell death. Thus only the TPS- and SYQ-peptides were used in subsequent studies to develop peptide grafted synthetic materials.

### Functional Characterization of Immobilized Peptides to Optimized Biomaterials in In Vitro Assays of Cell Binding.

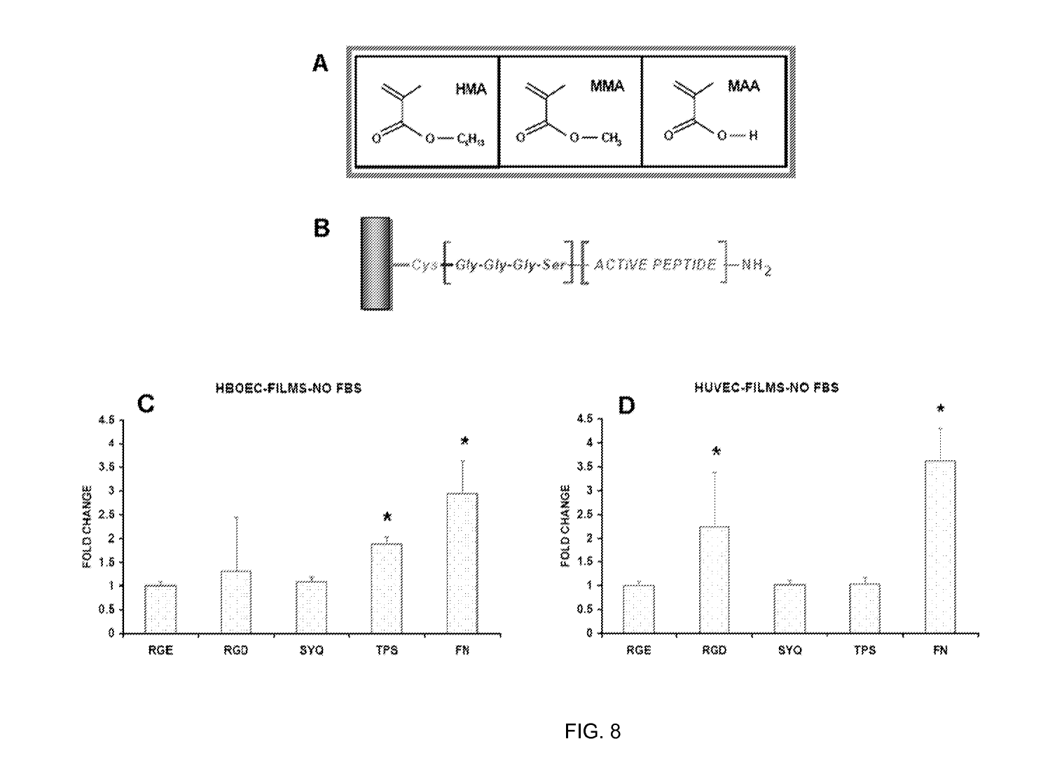

Phage display-selected peptide ligands were immobilized onto a methacrylic-based terpolymer matrix (FIGS. 8A, B). Methacrylates were chosen because of their widespread usage in biomedical applications and their ease of synthesis via free radical polymerization reaction. Free radical polymerization chemistry also provides a unique option for attaching peptide sequences using a chain transfer reaction (Fussell 2004a and Fussell 2004b). Phage display-selected ligands were incorporated into methacrylic terpolymers using a C-terminal cysteine residue as a chain transfer agent. FIG. 8C shows a bar graph quantifying HBOEC attachment to peptide-modified materials after 2 hours of incubation in a serum-free endothelial growth medium (EGM-2 Single Quotes, Cambrex). Peptide concentrations used were similar; approximately 2 μmol peptide per gram of terpolymer as determined by amino acid analysis. An RGE-containing terpolymer was used as a negative control to which HBOEC attachment was normalized. Glass cover slips coated with fibronectin were used as positive controls. The cell adhesive RGD peptide was also included in the study. Panel D in FIG. 8 represents results of HUVEC binding to the various peptide-modified substrates. While HUVEC attachment to RGD and fibronectin substrates was statistically significant compared to RGE negative controls, HUVEC did not attach to HBOEC-specific, peptide-modified substrates. The results from FIGS. 8C and D collectively show that the phage display-selected TPS-peptide was able to specifically modulate HBOEC binding when immobilized to a prosthetic surface. Other peptide sequences selected from the library screen are characterized in a similar manner for cell-specific binding to peptides conjugated to biocompatible surfaces.

### In Vivo Assay for Ligand Directed Endothelium Repair and Regeneration

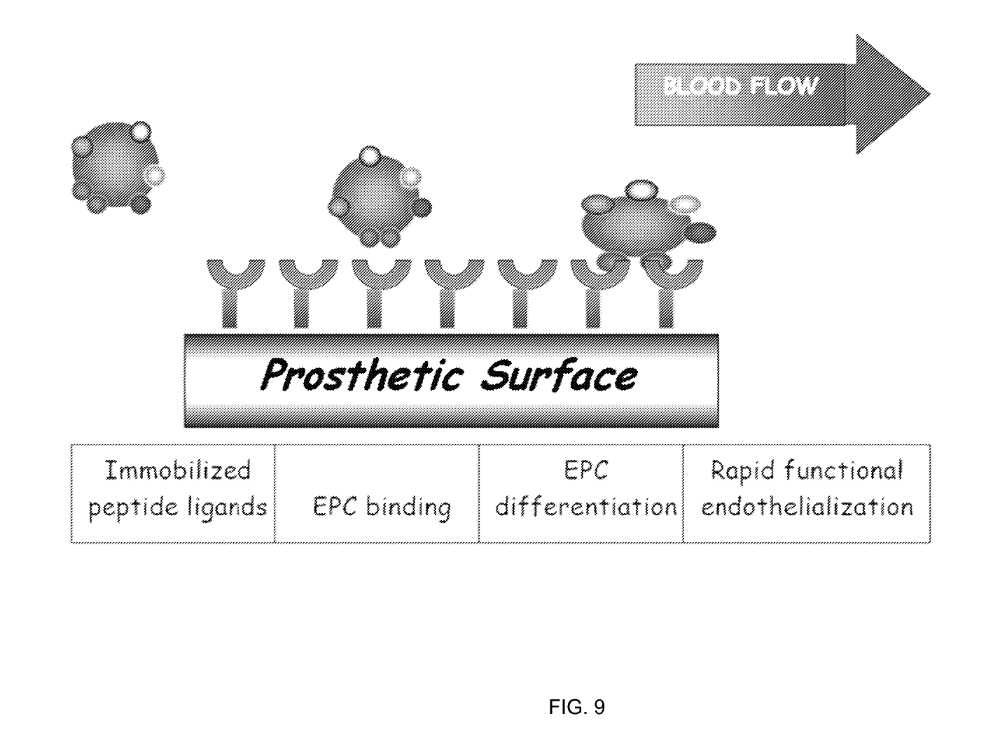

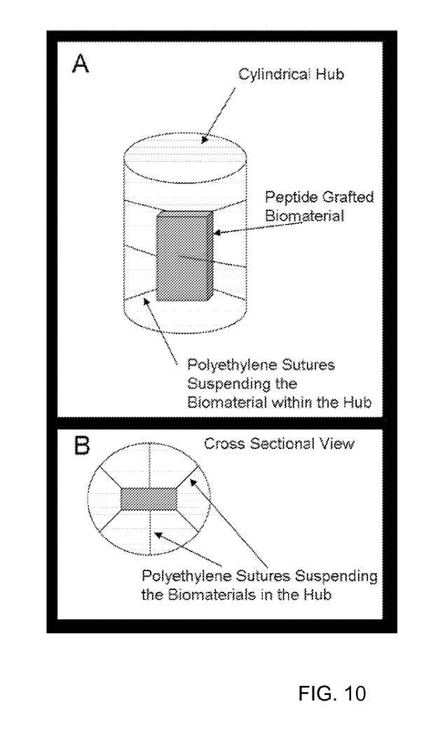

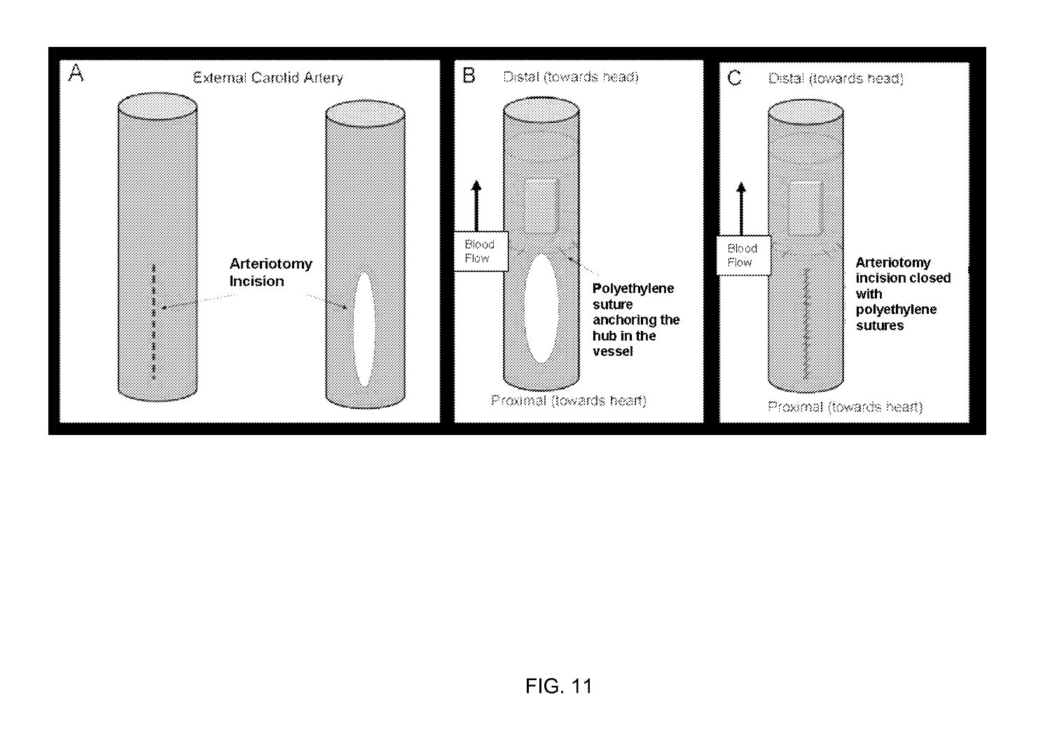

In vivo experiments are useful for triaging clinically useful peptides that bind with high affinity and specificity to circulating endothelial progenitor cells. Thus, additional studies are directed to whether the bioactive peptide-based materials provide a microenvironment for efficient attachment of endothelial progenitor cells, and to their differentiation and formation of functional endothelial monolayer (schematically illustrated in FIG. 9). While not bound by any particular theory or mechanism, endothelial progenitor cells from the peripheral circulation may be recruited to bioactive scaffolds that are functionalized with endothelial progenitor cell-specific ligands and these recruited cells may significantly contribute to enhanced endothelialization. A porcine carotid artery model is utilized to study endothelialization of implants as detailed in FIGS. 10 and 11. This experimental design eliminates the possibility of endothelial cells migrating from adjacent tissues, ensuring that the cells adhering to the test surface are derived from the flowing blood.

### Discussion

Phage display has proven to be a powerful strategy for the selection of peptides with desired binding properties (Hajitou et al. 2006; Rothe et al. 2006). The goal of the studies disclosed herein was to select peptide ligands that bind HBOEC with high affinity and specificity. The screening protocol resulted in the isolation of a panel of novel peptides and identification of several consensus motifs. Although no single peptide was sequenced from multiple clones, it has been previously shown that individual peptides isolated in this manner are efficient in binding their target in post phage display analysis (Cwirla et al. 1990; Koivunen et al. 1994). Small peptide motifs such as P(P/L)R, TP(T/S/G), PPS, and MPT appeared in these peptides. Although the overall significance of these peptides is unknown, the isolation of a number of peptides possessing identical motifs may be important in the binding of individual phage clones to the HBOEC surface. It has been suggested from other studies (Barry et al. 1996; Edelberg et al. 2004; Palmer et al. 1997; Szardenings et al. 1997) that peptides isolated from phage display often bind sites of protein-protein interaction raising the possibility that the HBOEC-binding peptides may target a functionally important binding site. A number of interesting homologies were identified (see Table 2), most notably interleukin-11 and ovarian cancer related tumor marker CA125 which may have implications for extending the understanding of HBOEC biology.

Phage selected through unbiased screens typically display specificity of 10-100 fold for the targeted cell type over other cell types (Oyama et al. 2006). The experiments disclosed herein confirm these observations. This finding is interesting when considering that the library was depleted only on HUVEC. While not bound by any particular theory or mechanism, this specificity suggests that the selected phage clones are not binding to a common receptor but may be targeting a less common receptor that is expressed on the target cell type.

TABLE 1
Peptide sequences identified by phage display.
Potential consensus motifs are indicated with
underlining. The primary consensus sequence,
obtained by most commonly observed amino acid at
each position, is presented in bold.

| PEPTIDE | SEQ ID NO: |
|---|---|
| HPAIVHISPQWA | 1 |
| QMVYGPLRSTEQ | 2 |
| NSLTSEPLRYGG | 3 |
| APFAHSGPLAFS | 4 |
| TPLHPKSLMVWH | 5 |
| SNSMHLMTMTGL | 6 |
| SVPPRYTLTLQW | 7 |
| TLDWTKPPLRSG | 8 |
| ASQGYPEHRHAS | 9 |
| HKSYLPVPSLYG | 10 |
| QTTKLHIMDTGF | 11 |
| ISPAPHLLTSRF | 12 |
| HGTNQALSLLTP | 13 |
| VLNPQTTVMPPL | 14 |
| ATTSLTPTMANH | 15 |
| QATGPTTPTTSG | 16 |
| TPSLEQRTVYAK | 17 |
| LYSASTPPDPGG | 18 |
| FPMSSYKTYATP | 19 |
| INTPANRNPVLG | 20 |
| WDTNRNAASTPG | 21 |
| SYQTLKQHLPYG | 22 |
| HHVDSLPTLDWK | 23 |
| KLPHQPPSAAVH | 24 |
| SPWTSFLQWARG | 25 |
| QFPPKLTNNSML | 26 |
| YTDNSLGTSVGK | 27 |
| TSLRELPAEWSR | 28 |
| SHGKPPSRSPWT | 29 |
| YNLGQLEAQITS | 30 |
| ITLSATKGAAPS | 31 |
| SPPPSNAGSHHV | 32 |
| THPPNPSVSIGG | 33 |
| MPTSSTAPPPLI | 34 |
| ANYFSSPIKHAT | 35 |
| HPPHNMHLPAFS | 36 |
| MPTLTRAPHTAC | 37 |
| LPRKTPDYLQTR | 38 |
| SPTPS(P/L)PPSAGG | 39 |

TABLE 2

Identified sequence homologies. Table showing potentially
relevant sequence homologies identified by BLAST. The underlined
amino acids correspond to exact matches in peptide sequence.

| | | |
|---|---|---|
| Peptide Sequence | Protein | Accession |

| | | |
|---|---|---|
| SVPPRYTLTLQW | Mucin glycoprotein (968-979) | AAQ82434 |
| HKSYLPVPSLYG | Transmembrane protein 2 (41-47) | CAI15172 |
| KLPHQPPSAAVH | T-cell specific adaptor protein (354-361) | AAF69027 |
| TPSLEQRTVYAK | 2700029M09Rik protein - glycoprotein (202-208) | AAH42740 |
| HHVDSLPTLDWK | Interleukin-11 (70-78) | NP_000632 |
| HHVDSLPTLDWK | Ovarian cancer related tumor marker CA125 (3732-3739) | AAL65133 |
| MPTLTRAPHTAC | Ovarian cancer related tumor marker CA125 (10670-10677) | AAL65133 |
| SPPPSNAGSHHV | Voltage dependent calcium channel (2040-2049) | CAI17142 |

The amino acid sequences represented by the accession numbers can be found in SEQ ID NO:40-46, respectively.

### REFERENCES

Altschul S F, Madden T L, Schaffer A A, Zhang J H, Zhang Z, Miller W, Lipman D J. 1997. Gapped BLAST and PSI-BLAST: a new generation of protein database search programs. Nucleic Acids Research 25(17):3389-3402.
Asahara T, Murohara T, Sullivan A, Silver M, vanderZee R, Li T, Witzenbichler B, Schatteman G, Isner J M. 1997. Isolation of putative progenitor endothelial cells for angiogenesis. Science 275(5302):964-967.
Ballard V L T, Holm J M, Edelberg J M. 2006a. A quantitative approach to phage display analysis for molecular profiling of vascular heterogeneity. Faseb Journal 20(4):A32-A32.
Ballard V L T, Holm J M, Edelberg J M. 2006b. Quantitative PCR-based approach for rapid phage display analysis: a foundation for high throughput vascular proteomic profiling. Physiological Genomics 26(3):202-208.
Barry M A, Dower W J, Johnston S A. 1996. Toward cell-targeting gene therapy vectors: Selection of cell-binding peptides from random peptide-presenting phage libraries. Nature Medicine 2(3):299-305.
Cwirla S E, Peters E A, Barrett R W, Dower W J. 1990. Peptides on Phage—a Vast Library of Peptides for Identifying Ligands. Proceedings of the National Academy of Sciences of the United States of America 87(16):6378-6382.
Edelberg J M, Wong A, Holm J M, Xaymardan M, Duignan I, Chin A, Kizer J R, Cai D Q. 2004. Phage display identification of age-associated TNF alpha-mediated cardiac oxidative induction. Physiological Genomics 18(3):255-260.
Fussell G. 2004a. Synthesis and characterization of acrylic terpolymers with RGD peptides for biomedical applications. Biomaterials. p 2971-2978.
Fussell G. 2004b. Endothelial cell adhesion on RGD-containing methacrylate terpolymers. Journal of biomedical materials research. p 265-273.
Gulati R, Jevremovic D, Peterson T E, Chatterjee S, Shah V, Vile R G, Simari R D. 2003a. Diverse origin and function of cells with endothelial phenotype obtained from adult human blood. Circulation Research 93(11):1023-1025.
Gulati R, Jevremovic D, Peterson T E, Witt T A, Kleppe L S, Mueske C S, Lerman A, Vile R G, Simari R D. 2003b. Autologous culture-modified mononuclear cells confer vascular protection after arterial injury. Circulation 108(12):1520-1526.
Gulati R, Simari R D. 2004. Autologous cell-based therapies for vascular disease. Trends in Cardiovascular Medicine 14(7):262-267.
Hajitou A, Pasqualini R, Arap W. 2006. Vascular targeting: Recent advances and therapeutic perspectives. Trends in Cardiovascular Medicine 16(3):80-88.
Iwakura A, Luedemann C, Shastry S, Hanley A, Kearney M, Aikawa R, Isner J M, Asahara T, Losordo D W. 2003. Estrogen-mediated, endothelial nitric oxide synthase-dependent mobilization of bone marrow-derived endothelial progenitor cells contributes to reendothelialization after arterial injury. Circulation 108(25):3115-3121.
Koivunen E, Wang B C, Ruoslahti E. 1994. Isolation of a Highly Specific Ligand for the Alpha(5)Beta(1) Integrin from a Phage Display Library. Journal of Cell Biology 124(3):373-380.
Lin Y, Weisdorf D, Solovey A, Hebbel R P. 2000. Origins of circulating endothelial cells and endothelial outgrowth from blood. Journal of Clinical Investigation 105(1):71-77.
Oyama T, Rombel I T, Sarnli K N, Zhou X, Brown K C. 2006. Isolation of multiple cell-binding ligands from different phage displayed-peptide libraries. Biosensors & Bioelectronics 21(10):1867-1875.
Palmer D B, George A J T, Ritter M A. 1997. Selection of antibodies to cell surface determinants on mouse thymic epithelial cells using a phage display library. Immunology 91(3):473-478.
Rehman J, Li J L, Orschell C M, March K L. 2003. Peripheral blood “endothelial progenitor cells” are derived from monocyte/macrophages and secrete angiogenic growth factors. Circulation 107(8):1164-1169.
Rothe A, Hosse R J, Power B E. 2006. In vitro display technologies reveal novel biopharmaceutics. Faseb Journal 20(10):1599-1610.
Smith G P. 1985. Filamentous Fusion Phage—Novel Expression Vectors That Display Cloned Antigens on the Virion Surface. Science 228(4705):1315-1317.
Smith G P, Petrenko V A. 1997. Phage display. Chemical Reviews 97(2):391-410. Szardenings M, Tornroth S, Mutulis F, Muceniece R, Keinanen K, Kuusinen A, Wikberg J E S. 1997. Phage display selection on whole cells yields a peptide specific for melanocortin receptor 1. Journal of Biological Chemistry 272(44):27943-27948.

### Example 2

### Methods

### Isolation and Characterization of HBOEC

The use of human blood samples was approved by ethical committee and institutional review board of the University of North Carolina, Chapel Hill. Blood was drawn from normal healthy individuals who gave informed consent to participate. Collected blood was added to buffered sodium citrate and subsequently diluted 1:1 with HBSS (Sigma-Aldrich, Milwaukee, Wis.) containing 1 mM EDTA and 0.5% BSA.

Mononuclear cells from the isolated blood were obtained by density gradient centrifugation as previously described³. Isolated mononuclear cells were cultured in EGM-2 complete medium (Cambrex Bioscience, Walkerville, Md.) and plated in a single well of 6-well culture dish (Costar, Lowell, Mass.) coated with type 1 Collagen (BD Biosciences, Bedford, Mass.). Non-adherent cells were removed 24 hrs after plating mononuclear cells and every second day thereafter; and HBOEC colonies with typical cobblestone morphology appeared within 3 to 4 weeks. Subsequently, HBOECs were expanded by standard cell culture techniques.

### Flow Cytometry

Flow cytometry of HBOEC: Cultured HBOECs were trypsinized and washed twice in PBS+0.1% BSA. Subsequently, HBOECs were then incubated with FcR blocking reagent (Miltenyi Biotech, Auburn Calif.). Cells were then washed and incubated with CD45-perCP (BD Bioscience, San Jose Calif.), CD34-APC (BD Bioscience, San Jose Calif.), CD31-FITC (BD Bioscience, San Jose Calif.), CD133-PE (Miltenyi Biotech, Auburn Calif.) or anti IL-11 receptor (K-20) antibody (Santa Cruz Biotechnology, Santa Cruz Calif.) conjugated with Alexa 488 (Invitrogen, Carlsbad, Calif.). Control HBOECs were incubated with appropriate isotype control antibodies. Thereafter, HBOEC were analyzed on Beckman Coulter (Dako) Cyan ADP flow cytometer. Flow cytometry data was analyzed by SummitV4.3 software.

Flow cytometry of mouse peripheral blood for CD34⁺/VEGFR2⁺ mononuclear cells: Approximately 1.0 ml of blood was drawn from Sv129 mice by cardiac puncture and immediately mixed with 1.0 ml of EGM-2 media with 0.5M EDTA. This mixture was subjected to Histopaque 1077 density-gradient centrifugation at 400 g at 23° C. for 30 min as previously described^{3,4}. At the end of the centrifugation, the buffy coat layer containing mononuclear cells was separated and treated with RBC lysis buffer. Cells were centrifuged and the pellet mononuclear cells which were resuspended in cold PBS+1% BSA. Cells were then aliquoted and their Fc receptors were blocked with rat anti-mouse CD16/CD32 antibody (BD Biosciences, San Jose Calif.). Cells were then incubated with primary antibodies against CD31 (BD pharmingen, San Jose, Calif.), CD34 (Abcam, Cambridge, Mass.), VEGFR2 (eBioscience San Diego, Calif.) and CD45 (BD pharmingen, San Jose, Calif.). Cells were analyzed on Beckman Coulter (Dako) Cyan ADP flow cytometer. Flow cytometry data was analyzed by SummitV4.3 software.

### Western Blot Analysis

Western blotting was performed as previously described^{5,6}. Briefly, HBOEC were incubated with 25 ng/ml recombinant human interleukin-11 (rhIL-11, R&D Systems, Inc., Minneapolis, Minn.; rhIL-11 corresponds to residues 22-199 of SEQ ID NO:44) for the indicated time. Cells were subsequently lysed in lysis buffer. Western blotting was performed by incubating primary antibodies against phosphorylated (Cell Signaling, Danvers, Mass.) and un-phosphorylated (Cell Signaling, Danvers, Mass.) forms of STAT-3. Blots were developed with an ECL kit (Amersham Pharmacia Biotech).

### Spheroid Angiogenesis Assay

HBOEC spheroids were generated as previously described⁷. Briefly, HBOEC were mixed with depleted EGM-2 growth media (Cambrex Bioscience, Walkerville, Md.) and carboxymethylcellulose (Sigma, Milwaukee Wis.). The slurry was incubated as hanging drops of 500 cells for 24 hrs in a cell culture incubator leading to aggregation of cells called spheroids. Spheroids were quickly added to a freshly prepared gel of neutralized collagen and carboxymethylcellulose at a ratio of 1:1. The spheroid containing gel was aliquoted into 24-well culture plates before the onset of gel polymerization. The mixture was incubated at 37° C. until polymerization occurred. Spheroids hung in the polymerized gel and produced sprouts in response to pro-angiogenic stimuli such as rhIL-11 or VEGF. The number and length of sprouts per spheroid were recorded to quantify angiogenic response. Images of the sprouts were taken at 10× magnification and imported into image J (National Institute of Health) to measure the number and cumulative length of sprouts per spheroid. For each experimental condition, at least 10 spheroids were analyzed.

### Cell Migration Assay

Cell migration assay of HBOEC was performed using 48-well chamber apparatus (NeuroProbe, Cabin John, Md.) as previously described⁸. Briefly, HBOEC were suspended in depleted EGM-2 medium in the upper chamber while various concentrations of rhIL-11 or VEGF were added to the lower chambers. The chamber was subsequently incubated for 4 hr and 30 min at 37° C. During the assay, HBOEC migrated across a collagen coated filter (8 μm pore size) from the upper chamber to the lower chamber. At the end of the assay, cells on the side of the filter facing the lower chamber were stained with hematoxylin and counted using a 10× objective on a Nikon Eclipse TS100 inverted microscope (Nikon, Melville, N.Y.). Four replicates of each well were used for each assay and four microscope observation fields were used from each well for calculations. Data is representative of three independent experiments.

### Unilateral Hindlimb Ischemia

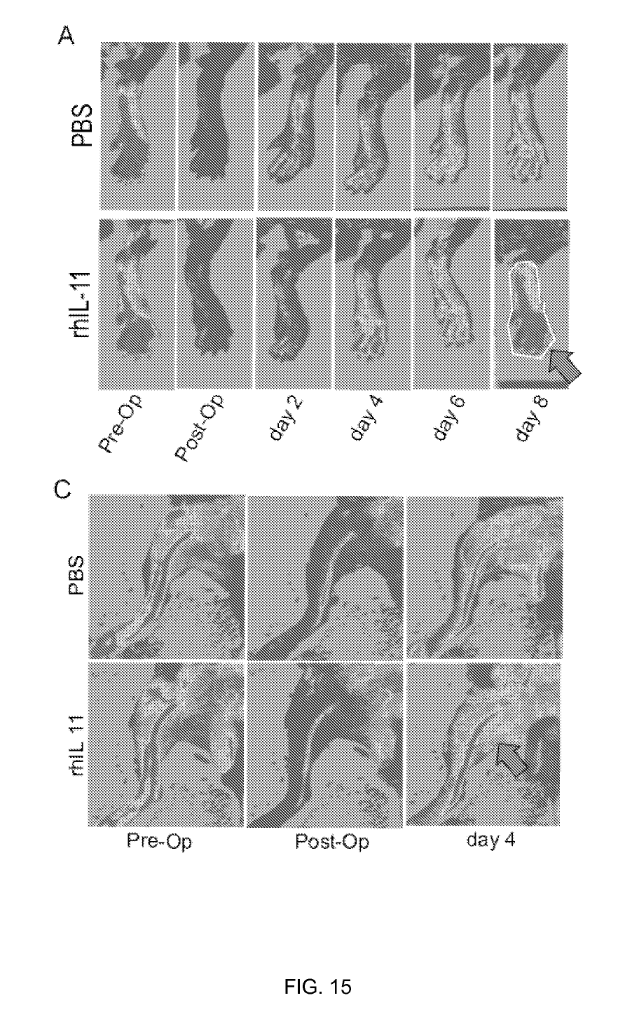

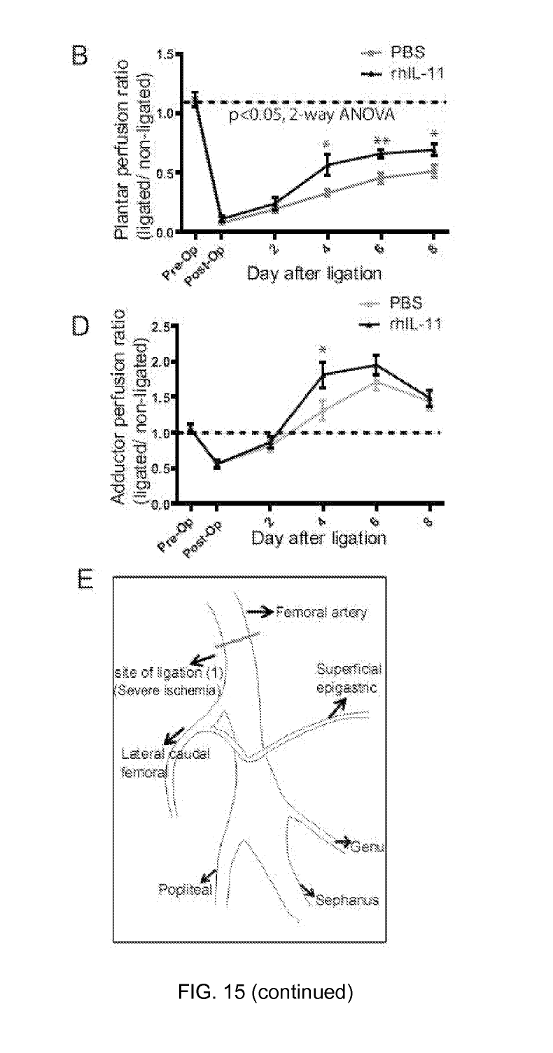

Unilateral femoral artery ligation was performed using 10-week old Sv129 mice as previously described^{9,10,11,12,13}. Briefly, mice were anesthetized with 1.25% isoflurane/O₂ during hindlimb depilation. In order to induce severe hindlimb ischemia, the right femoral artery was exposed through a 2 mm incision and ligated with two 7-0 sutures placed proximal to the origin of the lateral caudal femoral (see FIG. 15E). The artery was transected and separated. The wound was irrigated with sterile saline and closed, and Cefazolin (50 mg/kg, IM), Furazolidone (topical) and Pentazocine (10 mg/kg, IM) were administered. Procedures were approved by the University of North Carolina Institutional Animal Care and Use Committee.

### Laser Doppler Perfusion Imaging

Laser Doppler perfusion imaging was performed as previously described^{11,12,13}. Briefly, mice were anesthetized with 1.25% isoflurane/O₂ and their body temperature was strictly maintained at 37° C.±0.5 during the entire procedure. Perfusion imaging (Moor Instruments Ltd, Devon, UK) of plantar foot and adductor thigh of both legs was performed before, immediately after and at 2, 4, 6, and 8 days after femoral ligation. Perfusion images were analyzed using MoorLDIV5.0 software. The region of interest was drawn with respect to anatomical landmarks and flow rate was calculated. The ratio of flow rate in ligated/unligated leg was used for calculation. After femoral artery ligation, animals were individually inspected for foot appearance score [index of ischemia: 0, normal; 1-5, cyanosis or loss of nail(s), where the score is dependent on the number of nails affected; 6-10, partial or complete atrophy of digit(s), where the score reflects number of digits affected; 11, partial atrophy of forefoot]. Hindlimb use scores (index of muscle function) were also obtained: 0, normal; 1, no toe flexion; 2, no plantar flexion; 3, dragging foot.

### Results

Human Blood Outgrowth Endothelial Cells express IL-11Rα and administration of rhIL-11 activates downstream STAT-3

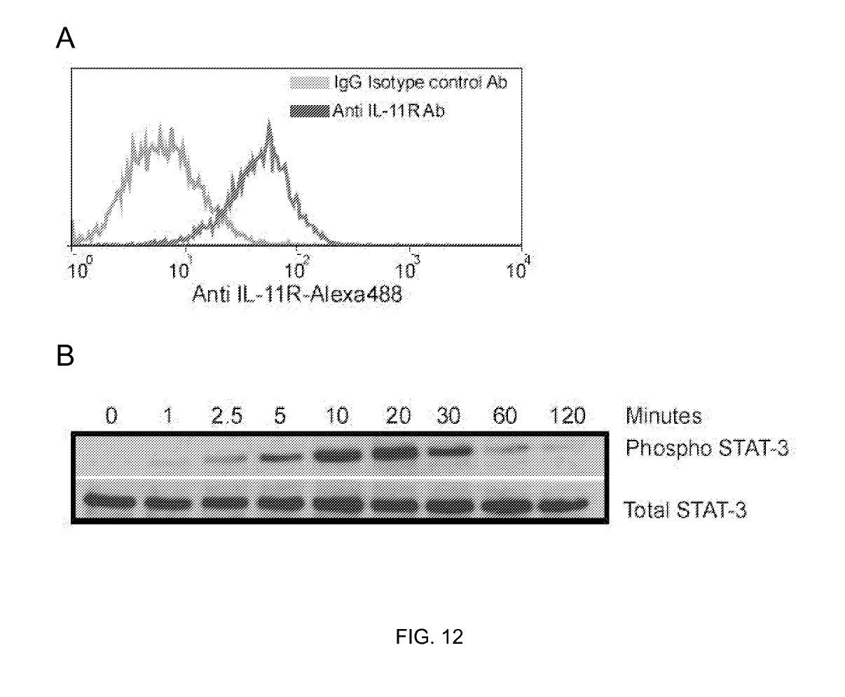

Peptide ligands that bind to HBOEC—cultured circulating endothelial cells³—were identified by phage display in Example 1. One of the 12-mer peptide ligands identified in Example 1 (SEQ ID NO:23) has sequence homology with human IL-11 (Table 2). This observation, coupled with reports of high expression of IL-11Rα in highly vascular tissues^{14,15,16,17} led to the investigation of the potential significance of IL-11/HBOEC interaction as it relates to vessel growth. As a first step, the phenotype of HBOEC was confirmed by multi-parametric investigation consisting of identification of their characteristic cobblestone morphology³ and acetylated LDL uptake¹⁷ as well as their expression of cell surface markers such as VEGFR2+ (a marker of endothelial cells, certain monocytes and hematopoietic precursors), CD34+ (a marker of hematopoietic precursors and endothelial cells, CD31+ (a marker of endothelial cells and monocytes) and lack of expression of CD133− (a marker that is present on hematopoietic precursors) and CD45− (a pan-hematopoietic marker)(data not shown)^{18,19, 1, 20,21}, suggesting that HBOEC have EPC properties^{22,23,24,25,26,27}. To determine if HBOEC express IL-11Rα, HBOEC were incubated with anti-IL-11 receptor antibody-Alexa488 (αIL-11R Ab-Alexa488) and it was found that HBOEC robustly express IL-11Rα receptors on their surface (FIG. 12A). In order to determine the functional significance of IL-11Rα/rhIL-11 receptor/ligand interaction, HBOEC were stimulated with rhIL-11 and it was found that rhIL-11 treated cells had a time-dependent phosphorylation of STAT-3 (FIG. 12B), indicating that IL-11Rα is a functional receptor on the surface of HBOEC and that IL-11Rα is activated and signals through downstream STAT-3 upon rhIL-11 stimulation.

### Recombinant Human IL-11 Stimulates Cell Migration and Sprouting Angiogenesis

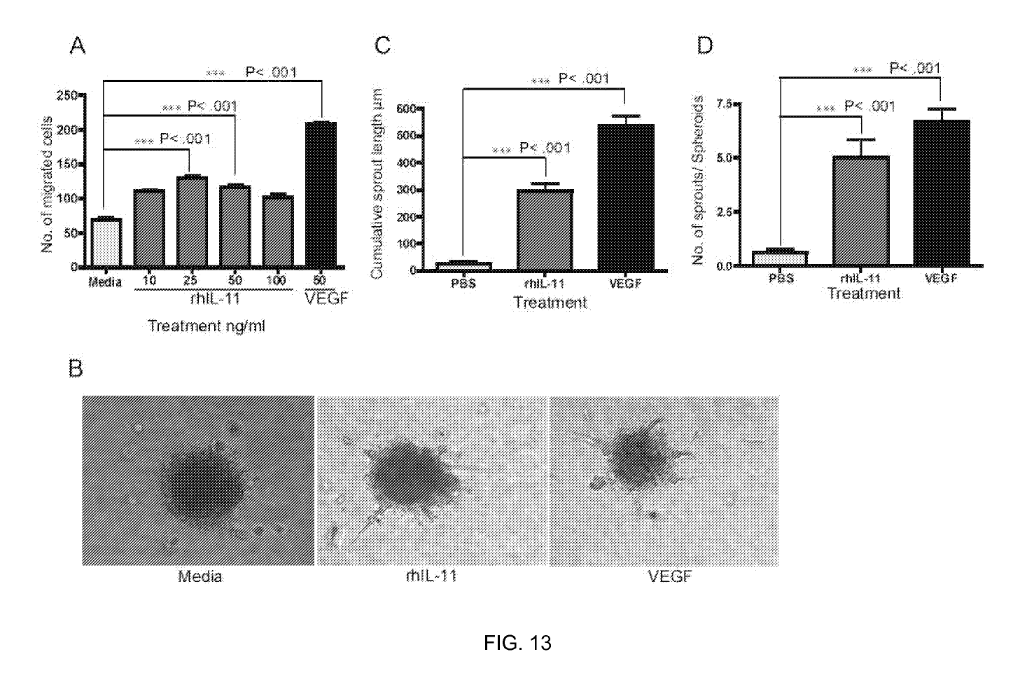

To study the physiologic role of rhIL-11, rhIL-11 was administered to HBOEC in a Boyden chamber at a concentration of 25 ng/ml. Recombinant human IL-11 administration led to cell migration of HBOEC towards a concentration gradient of rhIL-11 when compared to control (FIG. 13A). Similarly, rhIL-11 administration led to cell proliferation at a concentration of 50 ng/ml of rhIL-11 (data not shown). Since cell migration and cell proliferation are requisite for angiogenesis, we performed spheroid angiogenesis assays using HBOEC that were stimulated with rhIL-11 (FIG. 13B) and counted the cumulative sprout length and total number of sprouts for each spheroid. As shown in FIGS. 13C and 13D, rhIL-11 treated HBOEC exhibited 11-fold more cumulative sprout length (FIG. 13C) and 8-fold more sprouts/spheroids (FIG. 13D) when compared to HBOEC treated with PBS control. This observation showed that rhIL-11 has in vitro migratory effect on HBOEC and suggests a potential role of rhIL-11 for in vivo mobilization of progenitor cells.

In Vivo Mobilization of CD34⁺/VEGFR2⁺ Mononuclear Cells by rhIL-11

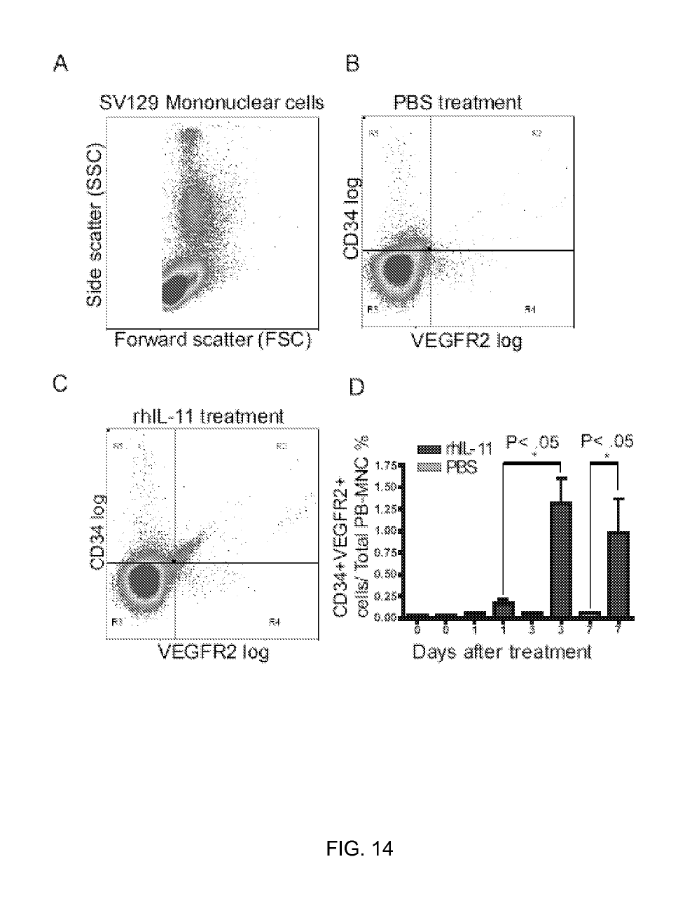

Shown herein is that HBOEC have EPC properties and that rhIL-11 administration has in vitro migratory effect on HBOEC. The potential role of rhIL-11 on in vivo mobilization of CD34⁺/VEGFR2⁺ mononuclear cells was investigated. Sv129 mice were implanted with an osmotic pump loaded with rhIL-11 or PBS to ensure continuous delivery of 200 μg/kg/day of rhIL-11 as previously reported²⁸. Blood was drawn from mice after 1 day, 3 days and 7 days after mini-pump implantation for mononuclear cell isolation and flow cytometry. FIG. 14A shows mononuclear cells profiled according to their forward and side scatter. Mice treated with rhIL-11 showed a significantly higher percentage of cells in the R2 quadrant which contains cells expressing both CD34+ and VEGFR2+ surface markers (FIG. 14C) when compared with PBS control (FIG. 14B). On the 3^{rd} day after rhIL-11 administration, there was a 20-fold increase in the number of CD34⁺/VEGFR2⁺ cells when compared with PBS treated mice (FIG. 14D), but this was not sustained during re-analysis on the 8^{th} day after femoral artery occlusion. This data shows that rhIL-11 can mobilize CD34⁺/VEGFR2⁺ mononuclear cells in vivo (presumably from the bone marrow) to peripheral circulation and provided the basis for further physiologic characterization of rhIL-11 as described below.

Increased Reperfusion after Femoral Artery Ligation in rhIL-11 Treated Mice

The observation that rhIL-11 treatment leads to rapid in vivo mobilization of CD34⁺/VEGFR2⁺ mononuclear cells (FIGS. 14C and D) prompted investigation of the physiologic significance of rhIL-11 and/or rhIL-11-mobilized CD34⁺/VEGFR2⁺ mononuclear cells on post-occlusive reperfusion and vascular remodeling. A mouse model of severe hindlimb ischemia was generated by surgical ligation of the right femoral artery of Sv129 mice proximal to the origin of lateral caudal femoral artery (FIG. 15E). This model of severe hindlimb ischemia does not utilize the lateral caudal femoral artery (in the adductor muscle) as a surrogate marker of perfusion as it is customarily reported^{9,10,11,12,13} since upstream ligation in the current model is proximal to this artery. Therefore, perfusion Doppler imaging of plantar vessels is a better index of perfusion in this model of severe hindlimb ischemia. High resolution infrared laser Doppler perfusion imaging (2 millimeter sampling depth) was used to measure perfusion in the plantar foot collateral vessel (which denotes aggregate of distal blood flow) at 2, 4, 6 and 8 days after femoral artery ligation. Immediately after femoral artery ligation, there was almost complete cessation of distal blood flow in the ligated limb of all mice. In addition, there was no significant change in distal blood flow in the ligated limb between PBS and rhIL-11 treated mice 2 days after femoral artery ligation (FIGS. 15A and 15B). This observation confirmed that all mice received proportional femoral artery ligation and suggests that rhIL-11 may not have acute effects on post-occlusive reperfusion. However, rhIl-11 treated mice showed a significant increase in plantar reperfusion and faster blood flow recovery at 4, 6 and 8 days after femoral artery ligation when compared with syngeneic mice treated with PBS under similar experimental conditions (FIGS. 15A and 15B). These observations suggest a novel role of rhIL-11 on collateral vessel reperfusion.

Although lateral caudal femoral artery perfusion imaging is not an ideal surrogate marker of distal perfusion in the present severe hindlimb ischemia model (for reasons explained above), a measure of adductor perfusion 2 days after femoral artery ligation showed no significant change in perfusion at this time point (FIGS. 15C and 15D) between rhIL-11 treated and control mice. However rhIL-11 treated mice showed significantly higher adductor perfusion at 4 days after femoral artery ligation compared to control mice (see FIG. 15D). Furthermore, the observed difference in perfusion was abrogated on day 6 and 8 after femoral artery occlusion, presumably due to enlargement of deeper adductor collaterals (beyond the sampling depth of the instruments employed in this study) in rhIL-11 treated mice, which may result in shunting of blood away from superficial vessels.

Improved Hindlimb Function in rhIL-11 Treated Mice

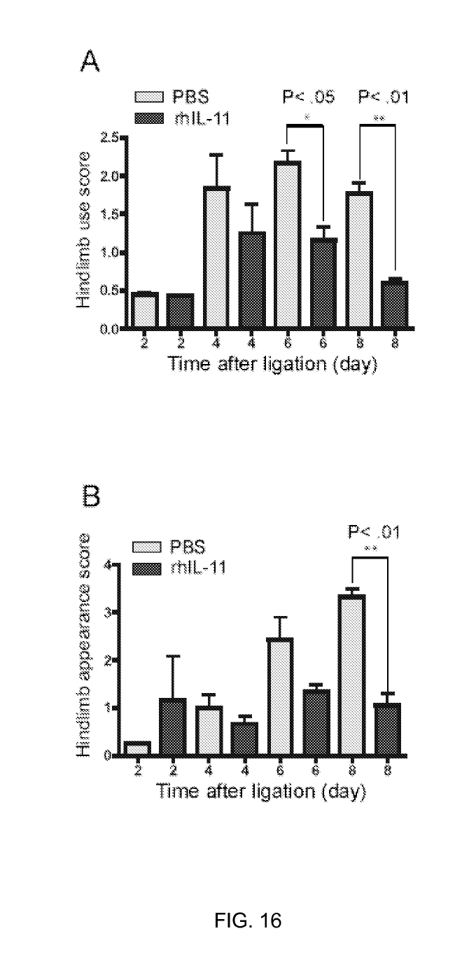

The observed increase in plantar perfusion in rhIL-11 treated mice prompted investigation of the temporal relationship between post-occlusive increased plantar perfusion and mouse hindlimb function. To this end, hindlimb use score (index of hindlimb muscle function) and hindlimb appearance score (index of hindlimb ischemia)^{9,10} were analyzed. rhIL-11 treated mice had better hindlimb use score and hindlimb appearance score (FIGS. 16A and B). Taken together, these data show that increased plantar perfusion (seen in rhIL-11-treated mice) is functionally significant as it correlates with functional recovery of hindlimb use.

### Interleukin-11 Regulates Collateral Vessel Growth and Remodeling

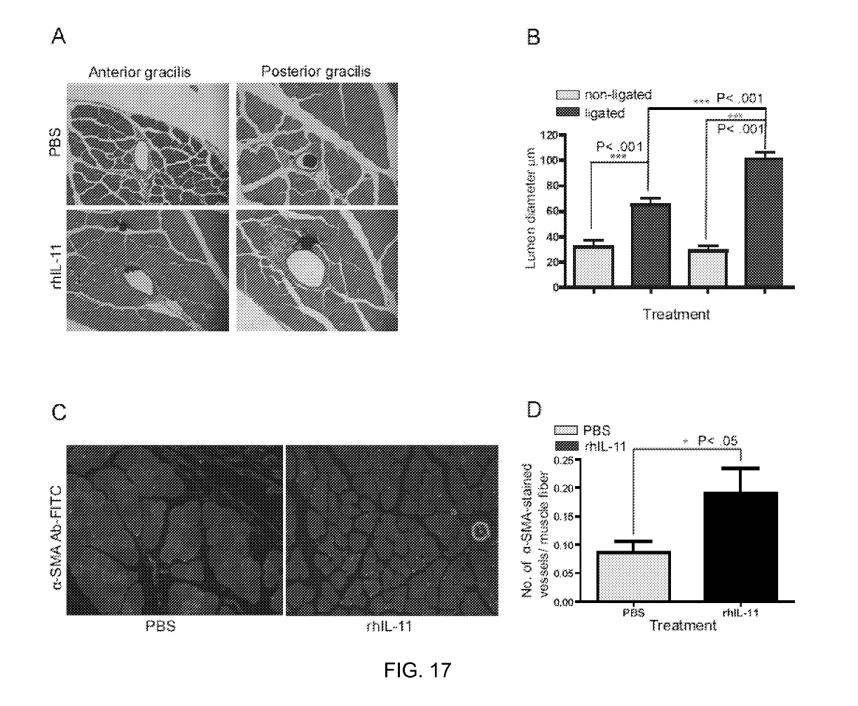

Collateral arteries are pre-existing arteriole-to-arteriole anastomoses (composed of endothelium and smooth muscle cells) that are recruited to restore blood flow and nutrients to distal vessels after arterial occlusion^{29,30,31}. Since increased collateral perfusion in rhIL-11-treated mice was observed, histologic sections were performed for morphometric analysis of collateral vessels in anterior and posterior gracilis muscle. As shown in FIGS. 17A and 17B, mice treated with rhIL-11 had 68% larger lumen diameter when compared to PBS control mice, suggesting that the observed increased perfusion in rhIL-11 treated mice is, at least partly, due to increased blood flow through large diameter collateral vessels. In an attempt to characterize the number of smaller conductance vessels, the number of alpha smooth muscle actin positive (α-SMA+) arterioles in the anterior and posterior gracilis muscle was counted as has been previously reported³². As shown in FIGS. 17C and 17D, rhIL-11 treated mice have 2.2-fold more α-SMA+arterioles than control mice. This data provides evidence that rhIL-11 regulates post occlusive collateral vessel remodeling.

### Interleukin-11 Regulates Homing of CD34⁺/VEGFR2⁺ Mononuclear Cells and Monocytes Toward Growing Collaterals.

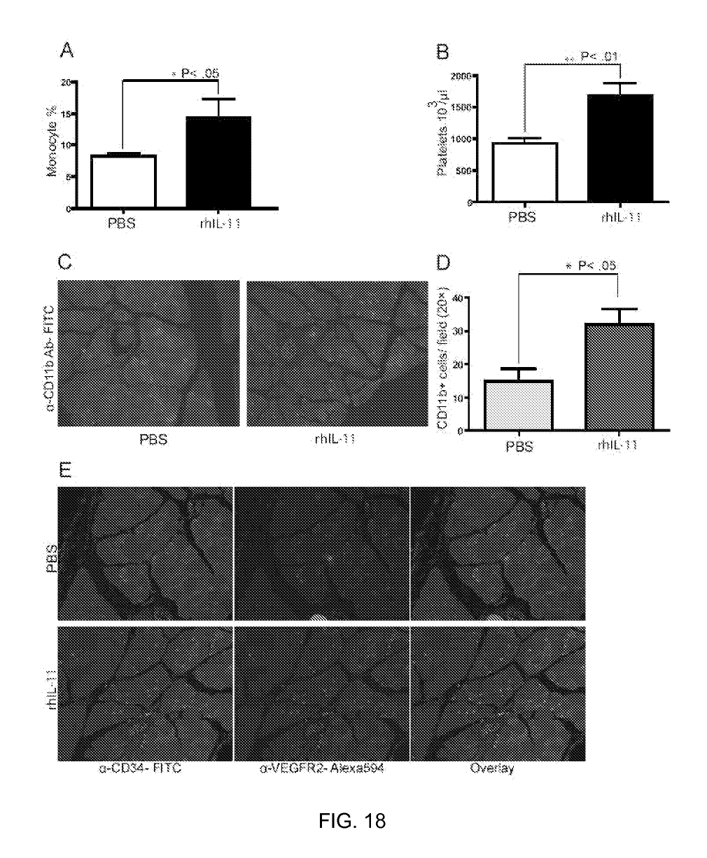

It has been previously reported that monocytes/macrophages and other bone marrow cells play a significant role in collateral vessel development^{33,34,35,36,37}. Since a differential increase in the number of circulating monocytes (FIG. 18A) and platelets (FIG. 18B) was observed with rhIL-11 treatment, it was hypothesized that rhIL-11-mediated collateral growth is functionally linked to mobilized CD34⁺/VEGFR2⁺mononuclear cells and monocytes. To this end, the number of CD34⁺/VEGFR2⁺ mononuclear cells and monocytes along growing collateral vessels in anterior and posterior gracilis muscle tissues were analyzed by staining this tissues for CD11b (Mac-1)—a marker of macrophages—and CD34/VEGFR2 (for CD34⁺/VEGFR2⁺ mononuclear cells) after the eighth post-operative day. As shown in FIGS. 18C and 18D, mice treated with rhIL-11 have a 2-fold increase in accumulation of monocytes/macrophages in the perivascular space when compared to control mice. Furthermore, rhIL-11 treated mice had significantly more perivascular CD34⁺/VEGFR2⁺ mononuclear cells when compared to control mice (FIG. 18E). While not bound by any particular theory or mechanism, this data suggests that rhIL-11 regulates collateral vessel remodeling by directing homing of CD34⁺/VEGFR2⁺ mononuclear cells and monocytes/macrophages toward the growing collateral vessels.

### Discussion

This data shows that rhIL-11 has a novel role in rapid in vivo mobilization of CD34+/VEGFR2+ mononuclear cells and monocytes into the peripheral circulation after femoral artery ligation of Sv129 mice. In addition, rhIL-11 administration leads to enhanced collateral vessel growth (arteriogenesis), increased collateral vessel perfusion, increased blood flow recovery and homing of CD34+/VEGFR2+ mononuclear cells and monocytes along growing collateral vessels suggesting a novel role of rhIL-11 in post-occlusive collateral vessel remodeling.

### REFERENCES

1. Rafii S, Heissig B, Hattori K. Efficient mobilization and recruitment of marrow-derived endothelial and hematopoietic stem cells by adenoviral vectors expressing angiogenic factors. Gene Therapy. 2002; 9:631-641.
2. Veleva A N, Stuart L, Patterson C. Selection and initial characterization of novel peptide ligands that bind specifically to human blood outgrowth endothelial cells. Biotechnology and Bioengineering. 2007; 98:306-312.
3. Lin Y, Weisdorf D J, Solovey A, Hobbel R P. Origins of circulating endothelial cells and endothelial outgrowth from blood. JCI. 2000; 105:71-77.
4. Somani A, Nguyen J, Milbauer L C, Solovey A, Sajja S, Hebbel R P. The establishment of murine blood outgrowth endothelial cells and observations relevant to gene therapy. Transl Res. 2007; 150:30-39.
5. Aitsebaomo J, Kingsley-Kallesen M L, Wu Y, Quertermous T, Patterson C. Vezf1/DB1 Is an Endothelial Cell-specific Transcription Factor That Regulates Expression of the Endothelin-1 Promoter. J. Biol. Chem. 2001; 276:39197-39205.
6. Aitsebaomo J, Wennerberg K, Der C J, Zhang C, Kedar V, Moser M, Kingsley-Kallesen M L, Zeng G-Q, Patterson C. p68RacGAP Is a Novel GTPase-activating Protein That Interacts with Vascular Endothelial Zinc Finger-1 and Modulates Endothelial Cell Capillary Formation. J. Biol. Chem. 2004; 279:17963-17972.
7. Korff T, Augustin H G. Integration of Endothelial Cells in Multicellular Spheroids Prevents Apoptosis and Induces Differentiation. J. Cell Biol. 1998; 143:1341-1352.
8. Ren R, Charles P C, Zhang C, Wu Y, Wang H, Patterson C. Gene expression profiles identify a role for cyclooxygenase 2-dependent prostanoid generation in BMP6-induced angiogenic responses. Blood. 2007; 109:2847-2853.
9. Chalothorn D, Zhang H, Clayton J A, Thomas S A, Faber J E. Catecholamines augment collateral vessel growth and angiogenesis in hindlimb ischemia. Am J Physiol Heart Circ Physiol. 2005; 289:H947-959.
10. Chalothorn D, Clayton J A, Zhang H, Pomp D, Faber J E. Collateral density, remodeling, and VEGF-A expression differ widely between mouse strains. Physiol. Genomics. 2007; 30:179-191.
11. Carmeliet P. Mechanisms of angiogenesis and arteriogenesis. Nature Medicine. 2000; 6:389-395.
12. von Degenfeld G, Banfi A, Springer M L, Wagner R S, Jacobi J, Ozawa C R, Merchant M J, Cooke J P, Blau H M. Microenvironmental VEGF distribution is critical for stable and functional vessel growth in ischemia. FASEB J. 2006; 20:2657-2659.
13. Clayton J A, Chalothorn D, Faber J E. Vascular Endothelial Growth Factor-A Specifies Formation of Native Collaterals and Regulates Collateral Growth in Ischemia. Circ Res. 2008; 103:1027-1036.
14. Campbell C L, Jiang Z, Savarese D M F, Savarese T M. Increased Expression of the Interleukin-11 Receptor and Evidence of STAT3 Activation in Prostate Carcinoma. Am J Pathol. 2001; 158:25-32.
15. Cork B A, Tuckerman E M, Li T C, Laird S M. Expression of interleukin (IL)-11 receptor by the human endometrium in vivo and effects of IL-11, IL-6 and LIF on the production of MMP and cytokines by human endometrial cells in vitro. Mol. Hum. Reprod. 2002; 8:841-848.
16. Zurita A J, Troncoso P, Cardo-Vila M, Logothetis C J, Pasqualini R, Arap W. Combinatorial Screenings in Patients: The Interleukin-11 Receptor {alpha} as a Candidate Target in the Progression of Human Prostate Cancer. Cancer Res. 2004; 64:435-439.
17. Urbich C, Dimmeler S. Endothelial Progenitor Cells: Characterization and Role in Vascular Biology. Circ Res. 2004; 95:343-353.
18. Duda D G, Cohen K S, Scadden D T, Jain R K. A protocol for phenotypic detection and enumeration of circulating endothelial cells and circulating progenitor cells in human blood. Nat. Protocols. 2007; 2:805-810.
19. Mallat Z, Silvestre J-S, Le Ricousse-Roussanne S, Lecomte-Raclet L, Corbaz A, Clergue M, Duriez M, Barateau V, Akira S, Tedgui A, Tobelem G, Chvatchko Y, Levy B I. Interleukin-18/Interleukin-18 Binding Protein Signaling Modulates Ischemia-Induced Neovascularization in Mice Hindlimb. Circ Res. 2002; 91:441-448.
20. Lin Y, Chang L, Solovey A, Healey J F, Lollar P, Hebbel R O. Use of blood outgrowth endothelial cells for gene therapy for hemophilia A. Blood. 2002; 99:457-462.
21. Wei J, Jarmy G, Genuneit J, Debatin K M, Beltinger C. Human blood late outgrowth endothelial cells for gene therapy of cancer: determinants of efficacy. Gene Ther. 2006; 14:344-356.
22. Rosell A, Arai K, Lok J, He T, Guo S, Navarro M, Montaner J, Katusic Z S, Lo E H. Interleukin-1[beta] augments angiogenic responses of murine endothelial progenitor cells in vitro. J Cereb Blood Flow Metab. 2009; 29:933-943.
23. Fan Y, Ye J, Shen F, Zhu Y, Yeghiazarians Y, Zhu W, Chen Y, Lawton M T, Young W L, Yang G-Y. Interleukin-6 stimulates circulating blood-derived endothelial progenitor cell angiogenesis in vitro. J Cereb Blood Flow Metab. 2007; 28:90-98.
24. Furstenberger G, von Moos R, Lucas R, Thurlimann B, Senn H J, Hamacher J, Boneberg E M. Circulating endothelial cells and angiogenic serum factors during neoadjuvant chemotherapy of primary breast cancer. Br J Cancer. 2006; 94:524-531.
25. Chakroborty D, Chowdhury U R, Sarkar C, Baral R, Dasgupta P S, Basu S. Dopamine regulates endothelial progenitor cell mobilization from mouse bone marrow in tumor vascularization. The Journal of Clinical Investigation. 2008; 118:1380-1389.
26. Povsic T J, Zavodni K L, Kelly F L, Zhu S, Goldschmidt-Clermont P J, Dong C, Peterson E D. Circulating Progenitor Cells Can Be Reliably Identified on the Basis of Aldehyde Dehydrogenase Activity. J Am Coll Cardiol. 2007; 50:2243-2248.
27. Friedrich E B, Walenta K, Scharlau J, Nickenig G, Werner N. CD34−/CD133+VEGFR-2+Endothelial Progenitor Cell Subpopulation With Potent Vasoregenerative Capacities. Circ Res. 2006; 98:e20-25.
28. Srivastava S, Charles P, Pi X, Moreno I, Veleva A, Hilliard E, Lockyer P, Patterson C, Aitsebaomo J. Abstract 3749: Interleukin-11 has a Novel Role in Proliferation and Migration of Cultured Endothelial Progenitor Cells in vitro And Mobilization of Endothelial Progenitor Cells in vivo. Circulation. 2008; 118:S_{—}480-
29. Fleisch M, Billinger M, Eberli F R, Garachemani A R, Meier B, Seiler C. Physiologically Assessed Coronary Collateral Flow and Intracoronary Growth Factor Concentrations in Patients With 1- to 3-Vessel Coronary Artery Disease. Circulation. 1999; 100:1945-1950.
30. Schaper W, Ito W D. Molecular Mechanisms of Coronary Collateral Vessel Growth. Circ Res. 1996; 79:911-919.
31. Sasayama S, Fujita M. Recent insights into coronary collateral circulation. Circulation. 1992; 85:1197-1204.
32. Potente M, Urbich C, Sasaki K-i, Hofmann W K, Heeschen C, Aicher A, Kollipara R, DePinho R A, Zeiher A M, Dimmeler S. Involvement of Foxo transcription factors in angiogenesis and postnatal neovascularization. The Journal of Clinical Investigation. 2005; 115:2382-2392.
33. Herold J, Tillmanns H, Xing Z, Strasser R, Braun-Dullaeus R. Isolation and transduction of monocytes: promising vehicles for therapeutic arteriogenesis. Langenbeck's Archives of Surgery. 2006; 391:72-82.
34. Bergmann C E, Hoefer I E, Meder B, Roth H, van Royen N, Breit S M, Jost M M, Aharinejad S, Hartmann S, Buschmann I R. Arteriogenesis depends on circulating monocytes and macrophage accumulation and is severely depressed in op/op mice. J Leukoc Biol. 2006; 80:59-65.
35. Imo E H, Sebastian G, Niels van R, Michiel V, Stephan H S, Susann U, Christoph B, Ivo R B, Jan J P. Leukocyte subpopulations and arteriogenesis: Specific role of monocytes, lymphocytes and granulocytes. Atherosclerosis. 2005; 181:285-293.
36. Heil M, Ziegelhoeffer T, Pipp F, Kostin S, Martin S, Clauss M, Schaper W. Blood monocyte concentration is critical for enhancement of collateral artery growth. Am J Physiol Heart Circ Physiol. 2002; 283(6):H2411-2419.
37. Ito W D, Arras M, Winkler B, Scholz D, Schaper J, Schaper W. Monocyte Chemotactic Protein-1 Increases Collateral and Peripheral Conductance After Femoral Artery Occlusion. Circ Res. 1997; 80:829-837.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for treating a vascular condition in a subject in need thereof comprising administering to said subject a therapeutically effective amount of a composition comprising interleukin-11.

2. The method of claim 1, wherein said interleukin-11 is selected from the group consisting of:
a) residues 22-199 of SEQ ID NO:44;
b) a variant of SEQ ID NO:44, wherein said variant has at least 80% sequence identity to residues 22-199 of SEQ ID NO:44, and wherein said variant is capable of recruiting or retaining outgrowth endothelial cells (OEC) at a therapeutic site of interest; and
c) a fragment of SEQ ID NO:44, wherein said fragment comprises at least 12 consecutive amino acids of SEQ ID NO:44, and wherein said fragment is capable of recruiting or retaining OEC at a therapeutic site of interest.

3. The method of claim 1, wherein said vascular condition is selected from the group consisting of atherosclerosis, peripheral vascular disease, heart disease, retinal vascular disease, myocardial infarction, stroke, thrombosis, peripheral artery disease, cerebrovascular ischemia, renal ischemia, pulmonary ischemia, limb ischemia, ischemic cardiomyopathy, and blood vessel injury.

4. The method of claim 1, wherein said subject is a mammal.

5. The method of claim 1, wherein said method further comprises administration of OEC to said subject.

6. The method of claim 5, wherein said OEC is bound to said interleukin-11.

7. The method of claim 1, wherein said composition further comprises one or more peptides selected from SEQ ID NO:1-39.

8. The method of claim 1, wherein the therapeutically effective amount is an amount sufficient to improve blood flow to an area affected by said vascular condition.

9. The method of claim 1, wherein the therapeutically effective amount is an amount sufficient to induce angiogenesis in an area affected by said vascular condition.

10. The method of claim 1, wherein the therapeutically effective amount is an amount sufficient to recruit or retain, or both, OEC in an area affected by said vascular condition

11. The method of claim 1, wherein interleukin-11 is administered at a therapeutic site of interest.

12. The method of claim 11, wherein said therapeutic site of interest is selected from the group consisting of an area where angiogenesis is desired, an area of ischemic injury, an area of organ transplantation, and an area of vascular injury.
